(19) United States
(12) Patent Application Publication
(10) Pub. No.: US 2019/0177719 A1
(43) Pub. Date: Jun. 13, 2019

(54) Method and System for Generating and Comparing Reduced Genome Data Sets

(71) Applicant: INSTITUTE FOR SYSTEMS BIOLOGY, Seattle, WA (US)

(72) Inventors: Gustavo Glusman, Seattle, WA (US); Robert Maxwell Robinson, Seattle, WA (US)

(21) Appl. No.: 16/306,706

(22) PCT Filed: May 26, 2017
(86) PCT No.: PCT/US17/34625

§ 371 Date: Dec. 3, 2018

Related U.S. Application Data
(60) Provisional application No. 62/344,329, filed on Jun. 1, 2016.
(60) Provisional application No. 62/411,165, filed on Oct. 21, 2016.

(51) Int. Cl. C12N 15/10; G16B 30/00; G16B 35/10; G16B 35/20; G16B 20/20

(52) U.S. Cl. CPC C12N 15/1089; G16B 30/00; G16B 20/20; G16B 35/20; G16B 35/10

(57) ABSTRACT

An ultra-fast solution to the problem of comparing genomes across sequencing technologies and genome freezes, while preserving privacy, is presented. A method for transforming a standard genome representation (i.e., a list of variants relative to a reference) into a “fingerprint” of the genome does not require knowledge of the technology, reference and encoding used, and yields fingerprints that can be readily compared to ascertain relatedness between two genome representations. Because of their reduced size, computation on the genome fingerprints is fast and requires little memory. This enables scaling up a variety of important genome analyses, including determinations of degree of relatedness, recognizing duplicative sequenced genomes in a set, and many others. Because the original genome representation cannot be reconstructed from its fingerprint, the method also has significant implications for privacy-preserving genome analytics.

### STATEMENT REGARDING FEDERALLY SPONSORED RESEARCH OR DEVELOPMENT

This invention was made with government support under grant NIH 1U54EB020406, awarded by the National Institutes of Health. The government has certain rights in the invention.

### FIELD OF THE DISCLOSURE

The present disclosure relates to new methods and systems for representing genome data and, more particularly, to new methods and systems for generation and analysis of reduced data sets representing genome data, and for facilitating analysis of genome data for comparison and relationship determination.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

The information in a genome is usually represented as raw genetic sequence and/or as a series of variants that are present in a genome, relative to a reference genome. A personal genome belonging to a human, for example, is represented as a series of variants from a corresponding human reference genome. Commonly, the reference genome is a public resource such as the genome sequence published as part of the Human Genome Project begun around 1990, declared complete in 2003, and improved steadily over the years since the first genome was sequenced. As a result, even for a single genome project, a number of reference genome versions (or “freezes”) exist, which differ, for example, by the inclusion of additional sequence where gaps existed in prior versions. The existence of multiple versions, and data reported relative to such versions, can make tracking of information and comparisons over time challenging.

Additionally, when enumerating variants relative to a single reference version, the encoding can be “zero based” or “one based.” That is, the first nucleotide of each chromosome is counted as position zero or one, respectively. Still further, a number of different sequencing technologies exist, and sequencing the same genome using different technologies can yield different results, as each technology has its own biases. Genomes can also be sequenced as a whole (whole genome sequencing) or in part (e.g., sequencing of one or more chromosomes or portions of chromosomes; exome sequencing; transcriptome sequencing).

For all of these reasons, one can have different representations of the genetic information from the same individual, and/or different annotations of the same genetic information. Given two representations of a genome, determining whether each is derived from the same individual can be a complicated procedure. A related problem is determining whether two genome representations are derived from related individuals (e.g., siblings, parent and child, etc.). These problems require knowledge about the technology, reference and encoding used. If these differ, comparing the genomes can be a slow, complicated, and error-prone bioinformatic procedure.

In addition to the complications above, comparative analysis of genomic information is time- and resource-intensive because of the huge volume of data in a genome: a haploid human genome contains more than three billion nucleotides.

Privacy considerations provide a further complication of genome analysis. While genetic information may be valuable to uniquely identify an individual, aspects of the genetic information can be associated with the existence of susceptibility to disease, and with a variety of other phenotypic traits. Applications exist where it would be helpful to retain the ability to identify an individual from genome sequence but anonymize or conceal phenotypic associations.

### SUMMARY

A method of generating a representation of a genome includes identifying for each single nucleotide variant (SNV) observed in a portion of the genome both a reference allele and a variant allele. The reference allele and the variant allele are joined together to form a SNV key for each single nucleotide variant in the portion of the genome. For each pair of consecutive SNVs, the method includes computing a variant-to-variant distance between the pair of consecutive SNVs, computing a reduced distance, creating a pair key, and incrementing a counting value corresponding to both the pair key and the reduced distance. In various embodiments, the portion of the genome may be the entire genome or a portion (e.g., a chromosome) of the genome. Computing the reduced distance may include finding a remainder after division of the variant-to-variant distance by a vector length, which vector length may be varied in different embodiments in order to adjust the specificity of the representation. Creating a pair key may include concatenating the SNV keys for each of the consecutive SNVs. Various embodiments also include normalizing the representation and/or adjusting the representation according to a selected population.

A method of comparing genetic information includes generating, from sequence data for a first genome, a first genetic fingerprint corresponding to the first genome. The method also includes generating, from sequence data for a second genome, a second genetic fingerprint corresponding to the second genome. Each of the genetic fingerprints identifies, for each of a set of pairs of consecutive SNVs in the sequence data for the respective genome, a number of pairs of SNVs having each of a plurality of particular reduced distances. A correlation is determined between the first and second genetic fingerprints. Determining the correlation between the first and second genetic fingerprints may include determining a Spearman correlation coefficient or a Pearson correlation coefficient, in embodiments. The correlation coefficient may be compared to one or more thresholds to determine a relationship between respective samples from which the sequence data of the first and second genomes were obtained.

The genetic fingerprints may be generated according to any of a variety of methods that include identifying for each SNV observed in the sequence data for the respective genome both a reference allele and a variant allele, joining the reference allele and the variant allele together to form a SNV key for each single nucleotide variant, and for each pair of consecutive SNVs, computing a variant-to-variant distance, the variant-to-variant distance between the pair of consecutive SNVs, computing a reduced distance, creating a pair key, and incrementing a counting value corresponding to both the pair key and the reduced distance.

The invention includes, as an additional aspect, all embodiments of the invention narrower in scope in any way than the variations defined by specific paragraphs above.

Where certain aspects of the invention are described as a genus or set, every member of the genus or set is, individually, an aspect of the invention. Likewise, every individual subset is intended as an aspect of the invention. By way of example, if an aspect of the invention is described as a members selected from the group consisting of 1, 2, 3, and 4, then subgroups (e.g., members selected from {1,2,3} or {1,2,4} or {2,3,4} or {1,2} or {1,3} or {1,4} or {2,3} or {2,4} or {3,4}) are contemplated and each individual species {1} or {2} or {3} or {4} is contemplated as an aspect or variation of the invention. Likewise, if an aspect of the invention is characterized as a range, such as a length range, then integer subranges are contemplated as aspects or variations of the invention.

The headings herein are for the convenience of the reader and not intended to be limiting. Additional aspects, embodiments, and variations of the invention will be apparent from the Detailed Description and/or Drawing and/or original claims.

Although the Applicant invented the full scope of the invention described herein, the Applicant does not intend to claim subject matter described in the prior art work of others. Therefore, in the event that statutory prior art within the scope of a claim is brought to the attention of the Applicant by a Patent Office or other entity or individual, the Applicant reserves the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 is a table depicting possible combinations of reference and variant alleles in single nucleotide variants (SNV keys) in an embodiment according to the present description;

FIG. 2 is a table depicting the possible combinations of two SNV keys of the table of FIG. 1;

FIG. 3 depicts a block diagram of an example computer and system programmed to implement a method or methods in accordance with the present description;

FIG. 4 is a flow chart depicting a first embodiment of a method for generating distance modulo fingerprints in accordance with the present description;

FIG. 5 is a flow chart depicting a second embodiment of a method for generating distance modulo fingerprints;

FIG. 6A is another embodiment of the concept of SNV keys depicted in FIG. 1;

FIG. 6B is yet another embodiment of the concept of SNV keys depicted in FIG. 1;

FIG. 6C is still another embodiment of the concept of SNV keys depicted in FIG. 1;

FIG. 7 is a flow chart depicting a method for normalizing distance modulo fingerprints;

FIG. 8 is a flow chart depicting a method for performing population adjustment on a distance modulo fingerprint;

FIG. 9 is a flow chart depicting a method for comparing distance modulo fingerprints;

FIG. 10 is a graph depicting the results of a study evaluating the strength of each vector length value for a particular set of genome data and a particular set of parameters and, in particular, a graph of vector length versus average and standard deviation for various levels of relatedness in a large family pedigree;

FIG. 11 is a flow chart depicting a first embodiment of a method for generating a binary distance modulo fingerprint according to the present description;

FIG. 12 is a flow chart depicting a second embodiment of a method for generating a binary distance modulo fingerprint;

FIG. 13 is a flow chart depicting a method for comparing binary distance modulo fingerprints;

FIG. 14 is table depicting an example embodiment of a distance modulo fingerprint;

FIG. 15A is a table depicting an example embodiment of genomic fingerprint masks;

FIG. 15B is a table depicting an example application of the genomic fingerprint masks of FIG. 15A to the first pair key (“ACAC”) from the fingerprint of FIG. 14;

FIG. 15C are a set of tables depicting an example application of the genomic fingerprint masks, as described for FIG. 15B, where the example application further includes an summation of the masks to the values of the pair key of the fingerprint to generate a binary digit encoding; and

FIG. 16 is a graph depicting prediction confidence as a measure fingerprint vector length versus standard deviation for the prediction, where various vector lengths for respective genomic fingerprints are used and those fingerprints' respective standard deviations are shown as the result of a comparison between fingerprints of known relationship types (e.g., siblings).

### DETAILED DESCRIPTION

A novel method and system for generating reduced genome data sets, and analyzing the reduced genome data sets to determine various relationships and parameters thereof, is described herein. Stylized as a “fingerprint” of the genome, the reduced data set is sufficiently distinct for a given individual that it can be compared to another such fingerprint to determine, based on the strength of correlations between the two, if the two are from the same person. However, unlike literal finger prints (i.e., the patterns of whorls and ridges in the tips of human fingers), the fingerprints described herein can also be used to determine the degree of relatedness of individuals, as well as other various parameters and characteristics, as will be elaborated upon below. A variety of other, additional advantages of the genomic fingerprints described below will become apparent throughout the remainder of the specification.

As used from this point forward, the term “fingerprint” refers to a set of data representing, for a whole genome or a portion of a genome, a reduced set of data representing a characterization of the distances between single nucleotide variants (SNVs), and optionally representing information about the successive variants. Exemplary portions of a genome, from which a fingerprint can be generated, include sequences of one or a subset of all of the chromosomes from a genome (e.g., the set of autosomes); sequences of substantial portions of a single or multiple chromosomes; exome sequences; and transcriptome sequences. For convenience, the invention is sometimes described herein by reference to “genome” or “exome,” with the intention and understanding that the method so described applies to practice of the method with other selected portions of genomes. For purposes of performing comparisons and analyzing correlations and relationships as described herein, it is important to compare fingerprints made with the same genetic information, e.g., comparison of a whole genome fingerprint with another whole genome fingerprint; or comparison of a whole Chromosome 1 fingerprint with another whole Chromosome 1 fingerprint; and so on. The term “distance” as applied to the distances between single nucleotide variants, is generally used to indicate the number of nucleotides between the two variants. Accordingly, two variants on consecutive positions have a distance zero, rather than 1. That is, the distance is not the difference between their positions, but instead is the number of intervening nucleotides. However, it is contemplated as within the scope of this disclosure that the distance could be measured as the distance between the coordinates of the variants and, as long as this is applied consistently, it would not change the overall function of the methods or systems herein described.

The invention described herein is especially useful in the context of analysis of the human genome. However, it can in principle also be used to generate and analyze/compare genomes of other animals or even organisms from other kingdoms, e.g., plants or fungi.

The phrase “distance modulo fingerprint” (or the abbreviation DMF) may refer to a specific type of fingerprint in which the reduced data set represents the frequency of consecutive single-nucleotide variants (SNVs), stratified at least by the modulus (i.e., the remainder after division) of the distance between them, and sometimes on the nature of the variations. This description focuses first on embodiments of this variety, while other embodiments will be described in later portions of the application, primarily because they are more easily understood once the earlier concepts are described. However, it should be understood that, more broadly, the phrase “distance modulo fingerprint” may refer to any fingerprint in which the reduced data set represents genetic data (e.g., data related to genotype, phenotype, etc.) in any manner described herein, that uses the modulo function to perform a hashing function on the data. By way of example, a DMF may be represented as a matrix having in one dimension (e.g., rows or columns) pairs of specific SNVs (e.g., a first SNV where the reference G allele changes to a variant A allele, followed by a second SNV where the reference G allele changes to a variant T allele), and in the other dimension (e.g., columns or rows) the possible modulus values (which are determined by a selected vector length; for example, a vector length 100 would be the modulus values possible for distance modulo 100, or 0 to 99). Various embodiments described herein result in DMFs represented by one-dimensional matrices, matrices that may or may not be related to distances between SNVs, matrices that are based, in part, on heterozygous alleles or homozygous alleles, and others, which will be clear in view of the remainder of the description.

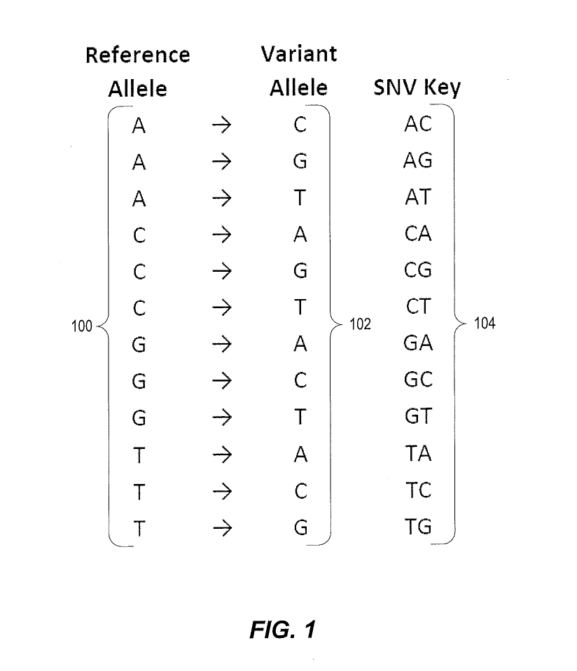

As alluded to above, in embodiments the fingerprints are generated, in part, according to the nature of the various SNVs occurring in a particular genome or portion of genome or exome, for example. As will be understood, the genetic information comprises sequences of four bases: adenine, cytosine, guanine, and thymine (in DNA) or uracil (in RNA) in various orders. In DNA the bases are present as deoxyribonucleosides (deoxyadenosine; deoxyguanosine; deoxythymidine; deoxycytidine). In RNA the bases are present as ribonucleosides (adenosine, guanosine, uridine, cytidine). For purposes of describing the fingerprints herein, the conventional abbreviations for the four bases (A, C, T, and G) are used, with the understanding that T in DNA is operationally equivalent to U in RNA for purposes of generating fingerprints. Many of these bases never, or rarely, vary between individuals in the same population (e.g., ethnicity) or in the same species. However, variations in specific positions or groups of positions are what differentiate one individual from another, and give each individual its unique characteristics and features. Given the four bases, there are 12 potential combinations of a reference allele 100 and a variant allele 102—as depicted in FIG. 1. (For RNA, each “T” in FIG. 1 represents a U.)

In some variations of the invention, genetic variations other than single nucleotide substitutions can be accounted for (considered) in generating the fingerprint, in which case SNV keys different from the twelve shown in FIG. 1 are used. For instance, four additional keys could be generated for single nucleotide deletions (AD, CD, GD, TD, where “D” represents deletion). Likewise, additional keys could be generated for single nucleotide insertions, and more complex variations, if desired. However, fingerprints suitable for uniquely identifying an individual and/or performing close relationship comparisons can be generated by consideration of only SNVs.

In some embodiments of the fingerprints, each SNV is represented by an SNV key 104 comprising the reference allele 100 followed by the variant allele 102. It follows, then, that a sequence of SNVs can be represented by a sequence of SNV keys 104. For example, if a first SNV has an SNV key AC (i.e., an A reference allele changed to a C variant allele), and a second, consecutive SNV has an SNV key AT (i.e., an A reference allele changed to a T reference allele), the pair of sequential SNVs can be represented by a pair key ACAT. In a similar manner, a triplet of sequential SNVs can be represented by a triplet key (e.g., ACATCG, for the pair of SNVs above, followed by an SNV with the SNV key CG). The pair keys (and triplet keys) represented in this manner are not sequences of consecutive nucleotides in this context, but rather, represent indications of pairs or groups of SNVs. That is, a triplet key is not a hexamer, as one might generally expect when seeing a sequence such as “ACATCG,” but instead represents three consecutive SNV keys that are separated by lengths of sequence that do not vary from the reference genome. Likewise, an SNV key (e.g., “AC”) does not represent two consecutive nucleotides, but instead represents the reference and variant alleles at the position of a single nucleotide.

It should be understood that the SNV keys depicted in FIG. 1 are but one possible set of SNV keys, the SNV keys are a way of representing specific variants, and may be represented in any of a variety of ways. For instance, each of the variations depicted in FIG. 1 could be numbered, and the SNV keys would be numbered. Alternatively, each of the variations depicted in FIG. 1 could be associated with a graphical symbol. In other embodiments, the reverse-complementary representation of SNV keys may be used. For example, the SNV key AC (i.e., an A reference allele changed to a C variant allele) may be represented instead as TG. In some embodiments, the reverse representation may be considered as equivalents to the original representations (e.g., AC), where such an equivalency representation would afford the flexibility of comparison in the presence of inversions or in the absence of a reference. In other embodiments, SNV keys may generated by encoding as the SNVs as either transitions or transversions (for achieving smaller fingerprints). SNV keys may also be generated by considering the dinucleotide or trinucleotide context of each SNV (for more detailed fingerprints).

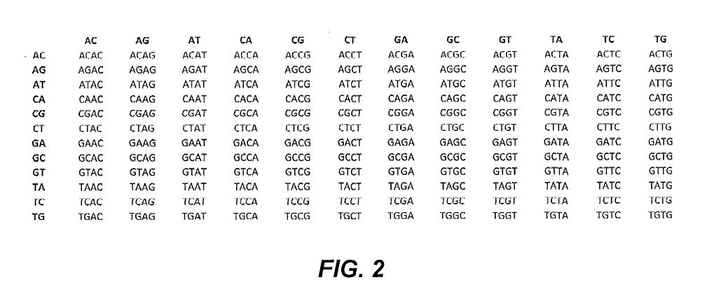

In some embodiments, the fingerprints are generated according to distances between pairs of SNVs and, accordingly, each pair of SNVs between which the distances are calculated may be represented by a corresponding pair key. FIG. 2 depicts the 144 possible pair keys that could be generated for pairs of SNVs represented by the SNV keys in FIG. 1. If, however, the SNV keys were numbered (e.g., 01, 02, . . . , 12 or 00, 01, . . . , 11), then the pair keys depicted in FIG. 2 would be sets of numbers, rather than letters, and if the SNV keys were symbols, the pair keys depicted in FIG. 2 would be pairs of symbols, with each symbol representing a specific SNV.

As will be understood, in a genome or portion of a genome being examined, the same pair key may be present many times. That is, a pair of SNVs with a given first SNV key (e.g., GA) followed by a given second SNV key (e.g., TC)—and, therefore, a pair key GATC—may occur in the genome or exome repeatedly. However, each occurrence of the two consecutive SNVs having SNV keys GA and TC may have a different intervening number of bases (i.e., a different intervening distance). For example, in a first occurrence of the SNV with SNV key GA followed by the SNV with SNV key TC, the two SNVs may be separated by a number, x, of bases, while in a second occurrence, the two SNVs may be separated by a number, y, of bases, where x and y may be different numbers.

As contemplated herein, in the distance modulo fingerprints, the pair keys are stratified according to the modulus of the distance between the SNVs that make up the pair key (the “distance modulo”). For a given vector length (i.e., a parameter selected according to the various goals and/or intended uses of the distance modulo fingerprints), the modulo function would yield as many “bins” into which pairs of SNVs could be “sorted.” By way of example and without limitation, for a vector length 20, each pair of SNVs could fall into one of 20 “bins” (represented as rows or columns, if the fingerprint is represented as a two-dimensional matrix). Each of the bins corresponds to the remainder of the distance divided by the vector length. For example, for a pair of SNVs having a distance of 100 bases, the pair would be in the 0 bin (100/20 yields a quotient of five with no remainder), while a pair of SNVs having a distance of 164 bases would be in the 4 bin (164/20 yields a quotient of eight with a remainder of four). Of course, those of ordinary skill in the art will appreciate that the number of bases between two SNVs is frequently hundreds or thousands, or even tens or hundreds of thousands, and that there will likely be a large number of bases between two SNVs forming a pair key.

Each DMF then represents, for each pair key, the number of times that pair key occurs in the genome or exome with a distance having each of the remainders for the selected vector length. Accordingly, in some embodiments, the DMF is stored and/or represented as a matrix of r rows, where r corresponds to the number of pair keys (and each row corresponds to a pair key) and c columns, where c corresponds to the vector length (and each column represents a specific remainder from 0 to one less than the vector length). Alternatively, the DMF is stored and/or represented as a matrix of r rows, where r corresponds to the vector length (and each row represents a specific remainder between 0 and the vector length) and c columns, where c corresponds to the number of pair keys (and each column corresponds to a pair key).

One should now be able to appreciate at least some of the benefits of the fingerprints and, in particular, the digital modulo fingerprints, relative to a genome sequence. Traditionally, a genome sequence requires information about all of the bases in the genome or exome, or at least about the position and variant of every single-nucleotide variant in the genome or exome. This is a significant amount of data, amounting to 735 MB for the human genome, which by some estimates can be losslessly compressed to about 4 MB. Even at 4 MB, automated (i.e., computer implemented) comparison of sequences can be a processor intensive and time-consuming process, especially when there are many hundreds or thousands of sequences that must be compared.

By contrast, fingerprints described herein (e.g., the distance modulo fingerprints and others) have a significantly smaller digital storage requirement. A distance modulo fingerprint implementing pair keys as described above, and using a vector length of 120, for example, can be compressed to a file size of 20-40 KB. An analysis of a set of genomes that would take one or several days using traditional representations of genetic sequences (e.g., to compare a small subset of data to a larger subset of data) can be accomplished with minutes for the entire set of data using the fingerprints herein described, while still providing much of the utility, as will be described below.

In some embodiments, which may or may not make use of the modulo function and which may be employed for particular purposes, each fingerprint can be compressed further, with some reducible to a 144 bit vector. Such embodiments will be described further below.

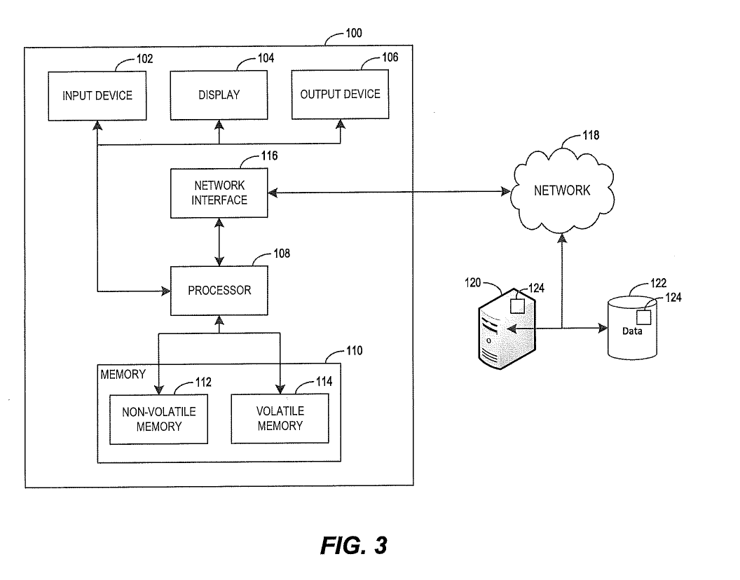

FIG. 3 depicts a block diagram of an example computer 100 and system programmed to implement a method or methods in accordance with the present description. The computer 100 includes one or more input device(s) 102, one or more display device(s) 104, one or more output device(s) 106, and one or more processor(s) 108. Each of the input devices 102 may be any known input device including, without limitation, a pointing device (e.g., a keyboard, a mouse, a track pad, a touch screen, etc.) that allows a user to operate and provide input to the computer 100. The input devices 102 may be internal (as in the case of a laptop computer) or external (as in the case of a USB mouse) to the computer 100, may be hard-wired to or removable from the computer, and may utilize any protocol that facilitates communication between the input device 102 and the processor(s) 108.

Similarly, the display(s) 104 and the output device(s) 106 may be internal (as in the case of a laptop display) or external (as in the case of a USB monitor or a printer), may be hard-wired to or removable from the computer, and may utilize any protocol that facilitates communication between the display(s) 104 and output device(s) 106 and the processor(s) 108. Of course, the displays 104 can utilize any known technology. Additionally, in embodiments, the display 104 may be coupled to and/or integrated with the input device 102, as would be the case in a touch-screen.

As will be understood, the processor(s) 108 may be one or more individual distinct processor packages, may be an integrated multi-core processor in a single package, or may even be multiple multi-core processor packages. The processor(s) 108 are programmed and/or programmable to perform the methods described below, according to machine readable instructions. The machine readable instructions may be stored on one or more memory device(s) 110 comprising any type of tangible, non-transitory media (e.g., magnetic media, solid state media, optical media, etc.) capable of storing data and/or machine-readable instructions executable by the processor 108. The memory 110 may have one or more elements of non-volatile memory 112 (e.g. solid state memory, hard drive, etc.) and one or more elements of volatile memory (e.g., Random Access Memory, or RAM) 114.

The processor 108 may also be communicatively coupled to a network interface 116. The network interface 116 is operable to communicate with one or more network devices via a communication protocol over a network 118. The network interface 116 may be communicatively coupled with the network 118 via any known (or later developed) wired or wireless technology, including without limitation, Ethernet networks, networks adhering to the IEEE 802.11 family of protocols, etc. The network 118, of course, may be any local or wide area network including, for example, the Internet, and may provide access to data (including machine-readable instructions, in embodiments) stored on one or more servers 120 and/or databases 122. In this manner, the processor 108 may retrieve, via the network interface 116 and the network 118, collections 124 of data stored on the servers 120 and/or the databases 122, which collections 124 of data may be updated periodically or in real time, in various embodiments. As a result, and as will be understood in view of the description to follow, the processor 108 may execute the methods described herein using the most recent collections 124 of data available as inputs, and/or may receive new data upon which to operate. Of course, data retrieved via the network 118 may be stored in either or both of the non-volatile memory 112 and the volatile memory 114 for later access and/or manipulation by the processor 108 and/or for comparison to current data stored on the servers 120 and/or the databases 122, in making a determination as to whether the one or more of the collections 124 of data have been updated since they were last retrieved via the network 118. The methods described herein may be stored in the volatile memory 114 and/or in the non-volatile memory 112.

The collections 124 of data stored on the servers 120 and/or the databases 122 may include, by way of example, various genetic sequence data. The data may include whole genome sequence data, exome sequence data, sequence data for a single chromosome, or even collections of single nucleotide polymorphisms, such as those generated by one or more SNP arrays. In embodiments, the collections 124 of data include collections of genetic sequence and/or SNP data that are generated using the same and/or different technologies as data in other collections or as other data in the same collection, the same and/or different encoding schemes as data in other collections or as other data in the same collection, the same and/or different labeling schemes as data in other collections or as other data in the same collection, the same and/or different reference freezes as other data collections or as other data in the same collection, etc.

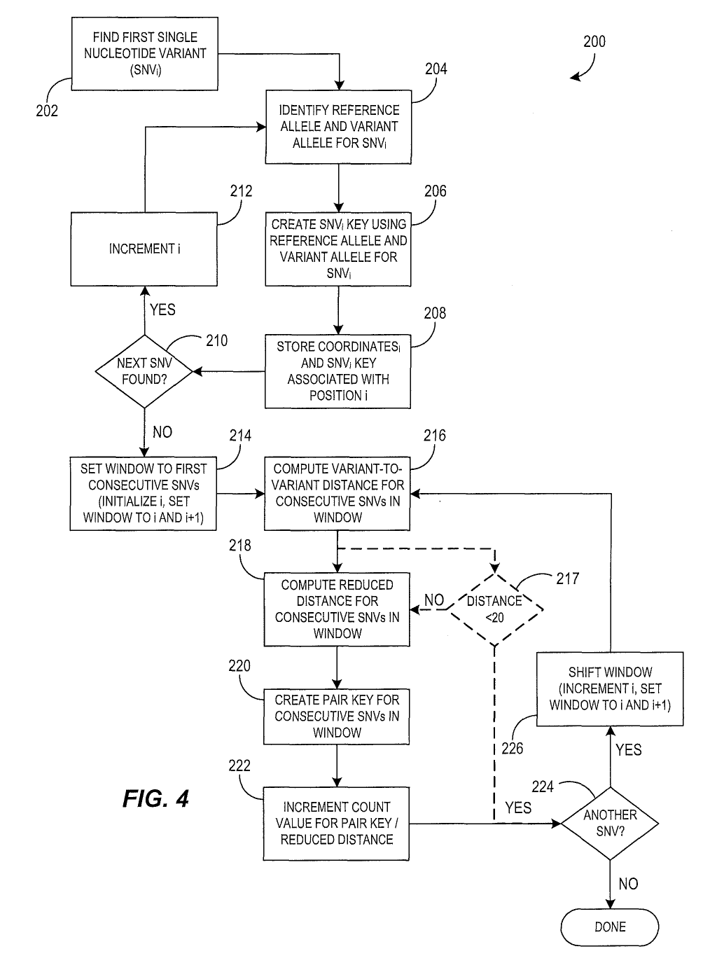

FIG. 4 depicts an embodiment of a first method 200 of generating Distance Modulo Fingerprints in accordance with the present disclosure. As described, the method 200 is performed by a computer processor (e.g., the processor 108) executing machine-readable instructions stored on a tangible, non-transitory computer readable medium (e.g., the memory 110). In the method 200, all of the available SNVs are located and stored before analysis for distances between them. However, in some embodiments of the method 200, the method may exclude non-autosomal variants (i.e., the method 200 may be applied only to autosomes). In other embodiments of the method 200, the zygosity (i.e., the number of haploid copies present) is considered. For example, Distance Modulo Fingerprints may be generated and compared using only heterozygous sites (i.e., variants where the genome is heterozygous). In such an embodiment, an advantage of minimizing reference bias and also comparison with fingerprints derived from de novo assemblies may be achieved.

In the method 200, the processor 108 determines the first SNV in the genetic sequence data under analysis (block 202). The genetic sequence data under analysis may be a whole genome sequence, a selected portion of a whole genome such as an exome sequence, or a series of SNPs. In any event, the genetic sequence data being analyzed may be stored in a digital file in the memory 110, or on a remote memory such as the server 120 or the database 122. The processor may retrieve the genetic sequence data (i.e., the file containing the data) and may therein locate the first SNV. The first SNV may be stored, for example, as SNVᵢ where i is the number is a value incremented to keep track of the ordinal position of the SNV relative to others being cataloged.

When the first SNV is located, the reference allele and the variant allele are determined (block 204). That is, relative to a particular reference genome or exome, at the location of the SNV, the alleles of the reference genome and the genome under analysis are noted. For example, if the reference genome has a “G” at the location of the SNV, and the genome under analysis has a “T” at the location of the SNV, the method would determine that the reference allele and the variant allele are G and T respectively, would create the SNV key for SNVᵢ using the reference and variant alleles (block 206). Thus, for the example above, the SNVᵢ key would be GT.

In some variations, the reference genome comprises genomic sequence data from a public resource such as the reference assembly prepared by the Genome Reference Consortium that has been improved steadily over the years since the first genome was sequenced. See https [colon-slash-slash] www.ncbi.nlm.nih.gov/projects/genome/assembly/grc/. Alternative reference genomes have been published and are likely to continue to be published and/or improved in the future, and are suitable for use as reference genomes for purposes described herein. (See, e.g., Krol, “The Hunt for a New Human Reference Genome,” published at http [colon-slash-slash] www.bio-itworld.com/2014/6/30/hunt-new-human-reference-genome.html; Steinberg et al., “Single haplotype assembly of the human genome from a hydatidiform mole,” Genome Res. (2014); 24: 2066-2076 (incorporated herein by reference); and http [colon-slash-slash] www.ncbi.nlm.nih.gov/sra) under project SRP017546. The GenBank Assembly ID for the CHM1_1.1 assembly described by Steinberg is GCA_000306695.2.) Due to the increasing ease and decreasing cost of sequencing, it also is possible to use a customized public or private reference genome. For instance, a reference genome can be constructed based on whole genome sequencing of members of a population selected by one or more phenotypic traits, by geographic origin, by cultural or racial or ethnic origin (as self-identified by the subjects and/or as identified by one or more genetic markers selected as representative of a racial or ethnic population).

Additionally, in lieu of sequencing, it is possible to characterize the identity of hundreds, or thousands, tens of thousands, hundreds of thousands, or millions of SNVs using hybridization analysis of a subject's nucleic acid using DNA microarrays of immobilized, allele-specific oligonucleotide probes (“SNP chips”) designed to detect and identify alleles of single nucleotide polymorphisms (SNP). Extensive public libraries of SNP information exist from which it is possible to discern both the identity of SNP alleles (wild type or reference version of a SNP and variants thereof) and the location in the human genome. See, e.g., http[colon-slash-slash]www.ncbi.nlm.nih.gov/snp or http[colon-slash-slash]www.ncbi.nlm.nih.gov/projects/SNP/or http[colon-slash-slash]www.uniprot.org/database/DB-0013 or http[colon-slash-slash] www.hgvs.org/central-mutation-snp-databases. Thus, a dataset comprised of SNP array data can be used as a reference genome for purposes described herein, and SNP arrays can be used to obtain SNV information for the genome to be analyzed.

Generally speaking, when a data set is selected as the reference genome, then single nucleotide variations from the data set are the SNVs. If a reference genome is being constructed from data generated from a plurality of genome sequences, then typically the more prevalent allele of a SNP is designated as the reference allele, and less prevalent alleles are scored as the variant version.

The coordinates of SNVᵢ, as well as the SNVᵢ key are stored as associated with ordinal position i (block 208). These data may be stored in a table, for example, having one row for each SNV, and in each row having the coordinates of the SNV in the genome and the SNV key associated with the SNV. Of course, there are many ways that the data could be stored. The method 200 continues by looking for additional SNVs. If another SNV is found (block 210), then the value of i is incremented (block 212), and the method repeats blocks 204, 206, 208.

In some variations, the method is practiced with respect to a single contiguous polynucleotide, such as a single chromosome, in which case all of the SNVs have a measurable distance from an adjacent SNV. In some variations, the method is practiced with respect to a genome or portion of a genome than includes two or more discrete polynucleotides. (For instance, each human diploid cell normally contains 23 pairs of chromosomes, for a total of 46 chromosomes, and each chromosome is a discrete polynucleotide.) In variations involving two or more polynucleotides, the method steps 202, 204, 206, 208, 210, 212 are repeated for each discrete polynucleotide. (The coordinates of the last SNV that occurs on one polynucleotide need not be compared with the first SNV that occurs on a successive discrete polynucleotide.)

If no more SNVs are found (block 210), indicating that the genome or exome being analyzed has no further SNVs, then the set of SNVs is analyzed. This may be accomplished by a windowing method. For example, a window of two consecutive SNVs on the same chromosome may be analyzed. The window may first be set to the first two consecutive SNVs in the data (i.e., SNVᵢ and SNVᵢ₊₁ when i is set to its initial value) (block 214). For SNVᵢ and SNVᵢ₊₁, the associated coordinate data are retrieved from memory and, using the coordinates, the variant-to-variant distance (e.g., in terms of the number of bases between the two SNVs, which must be on the same chromosome) is determined or computed (block 216). That distance may, in embodiments, be reduced using the modulo function to generate a remainder value for the distance between the SNVs, which remainder value will be the value associated with the pair of SNVs in the window (block 218). The remainder value will be the distance modulo the vector length, which is determined, in some embodiments, according to various parameters, including, for example, the amount of specificity desired in the distance modulo fingerprint data. In other embodiments, the SNV pair distances may also be reduced by other means including, but not limited to, any of scaling linearly and either winsorizing or ignoring distances above a threshold (and where the relevant parameters become the scaling factor and the maximal value, in place of the vector length as used in the modulo strategy); scaling using a nonlinear function like log or square root; or binning using variable bin sizes to account for the observed distribution of SNV distances observed in collections of genomes.

In various embodiments, the vector length is 20, 50, 100, 120, 150, or 200. In various embodiments, the vector length is between 2 and 200, between 10 and 25, between 2 and 25, between 2 and 50, between 10 and 50, between 50 and 100, between 50 and 150, between 100 and 150, between 100 and 125, between 125 and 150, between 100 and 200, between 150 and 200, or between 2 and 125. Accordingly, the distance (expressed as the number of bases) between the two SNVs in the window is divided by the vector length, and the remainder determined. (By way of example, for a vector length of 20, a distance of 153 and a distance of 140,133, would both have a “reduced distance” (i.e., a remainder) of 13. Every number would have a reduced distance between 0 and 19, inclusive.)

For each window (e.g., each window SNVᵢ and SNVᵢ₊₁), the SNV keys associated with the SNVs in the window may be concatenated to create a pair key (block 220). If SNVᵢ had an SNV key GT, and SNVᵢ₊₁ had an SNV key CT, for example, the method 200 would create a pair key GTCT. For a window length of two, where each SNV key is created using only the reference and variant alleles, then, there are 144 possible pair keys, as depicted in FIG. 2. Of course, while described herein as concatenations of the SNV keys, the pair key is simply a symbolic representation related to the SNV, and need not be a concatenation of SNV keys. The pair keys could be the joined SNV keys with a delimiter (e.g., “GT,CT”), or even any alphanumeric or graphical symbol associated with each particular pair of SNVs.

All of these data may be stored, for example, in a table that has a row for each pair key (e.g., 144) and a column for each possible reduced distance (e.g., a number of columns equal to the vector length). In such a table, the cell that corresponds to the row for the pair key and the column of the reduced distance, can contain a value that indicates the number of times the pair of consecutive SNV keys has been separated by a number of base pairs that, when divided by the vector length, results in a remainder of the value associated with that particular column. (Of course, the rows and columns can be reversed—the rows corresponding to the reduced distances and the columns corresponding to the pair keys—without requiring any significant experimentation by the person implementing the method 200.) Thus, for the window including SNVᵢ and SNVᵢ₊₁, the value corresponding to the pair key and the reduced distance is incremented (block 222). If another SNV exists (block 224), the window is shifted (i is incremented and the window again set to SNVᵢ and SNVᵢ₊₁) (block 226), and blocks 216, 218, 220, 222, and 224 are repeated. If no more SNVs exist, the method 200 of determining the digital genomic fingerprint for the set of data is complete.

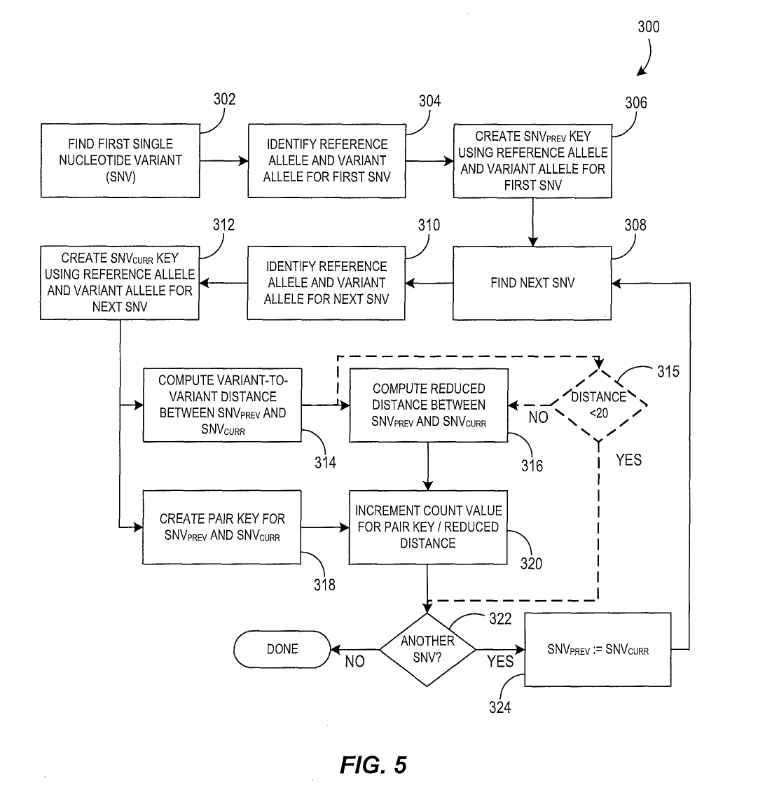

FIG. 5 illustrates an alternate embodiment of a method 300 for generating the distance modulo fingerprints. As described, the method 300 is performed by a computer processor (e.g., the processor 108) executing machine-readable instructions stored on a tangible, non-transitory computer readable medium (e.g., the memory 110). In the method 300, the available SNVs are located and processed one at a time with reference to the SNV immediately preceding.

In the method 300, the processor 108 determines the first SNV in the genetic sequence data under analysis (block 302). The genetic sequence data under analysis may be a whole genome sequence, a portion of a whole genome such as an exome sequence, or a series of SNPs. In any event, the genetic sequence data being analyzed may be stored in a digital file in the memory 110, or on a remote memory such as the server 120 or the database 122. The processor may retrieve the genetic sequence data (i.e., the file containing the data) and may therein locate the first SNV.

When the first SNV is located, the reference allele and the variant allele are determined (block 304). That is, relative to a particular reference genome or exome, at the location of the SNV, the alleles of the reference genome and the genome under analysis are noted. For example, if the reference genome has a “G” at the location of the SNV, and the genome under analysis has a “T” at the location of the SNV, the method would determine that the reference allele and the variant allele are G and T respectively, would create the SNV_{PREV} key for the SNV using the reference and variant alleles for the first SNV, and store it with the coordinates of the first SNV (block 306). Thus, for the example above, the SNV_{PREV} key would be GT.

The method 300 continues when the processor 108 engages in finding the next (current) SNV (block 308), identifying the reference allele and variant allele for the next SNV (block 310), creating an SNV_{CURR} key using the reference and variant alleles (block 312) of the current SNV and storing it with the coordinates of the current SNV. The processor 108 then computes retrieves the associated coordinate data from memory and, using the coordinates, computes or determines the variant-to-variant distance (e.g., in terms of the number of bases between the two SNVs) between SNV_{PREV} and SNV_{CURR} (block 314). That distance may, in embodiments, be reduced using the modulo function to generate a remainder value for the distance between the SNVs, which remainder value will be the value associated with the pair of SNVs in the window (block 316). As in the method 200, the remainder value will be the distance modulo the vector length, which is determined, in some embodiments, according to various parameters, including, for example, the amount of specificity desired in the distance modulo fingerprint data.

As above, in various embodiments, the vector length is 20, 50, 100, 120, 150, or 200. In various embodiments, the vector length is between 2 and 200, between 10 and 25, between 2 and 25, between 2 and 50, between 10 and 50, between 50 and 100, between 50 and 150, between 100 and 150, between 100 and 125, between 125 and 150, between 100 and 200, between 150 and 200, or between 2 and 125. All integer values between 2 and 500 are specifically contemplated as vector lengths suitable for practice of the invention. Accordingly, the distance (expressed as the number of bases) between the two SNVs in the window is divided by the vector length, and the remainder determined. (By way of example, for a vector length of 20, a distance of 153 and a distance of 140,133, would both have a “reduced distance” (i.e., a remainder) of 13. Every number would have a reduced distance between 0 and 19, inclusive.)

The processor 108 executing the method 300 also creates a pair key for the pair of SNVs SNV_{PREV} and SNV_{CURR} (block 318). All of these data—the reduced distance between the SNVs represented by SNV_{PREV} and SNV_{CURR}, and the pair key for SNV_{PREV} and SNV_{CURR}—may be stored, for example, in a table that has a row for each pair key (e.g., 144) and a column for each possible reduced distance (e.g., a number of columns equal to the vector length). In such a table, the cell that corresponds to the row for the pair key and the column of the reduced distance, can contain a value that indicates the number of times the pair of consecutive SNV keys has been separated by a number of base pairs that, when divided by the vector length, results in a remainder of the value associated with that particular column. (Of course, the rows and columns are simply different dimensions of the data and can be reversed—the rows corresponding to the reduced distances and the columns corresponding to the pair keys—without requiring any significant experimentation by the person implementing the method 300.) Thus, after computing the reduced distance and creating the pair key for SNV_{PREV} and SNV_{CURR}, the processor 108 may increment the count value in the cell corresponding to the pair key and the reduced distance (block 320). If another SNV exists (block 322), the data for SNV_{PREV} are set equal to the data for SNV_{CURR} (block 324), and blocks 308, 310, 312, 314, 316, 318, 320, and 322 are repeated. If no more SNVs exist, the method 300 of determining the digital genomic fingerprint for the set of data is complete.

As described above, SNV_{PREV} and SNV_{CURR} are concepts relevant to computing the distances between SNVs on a single polynucleotide chain. For genomes with two or more polynucleotide chains, the routine is repeated for each chain, with the results being accumulated on the same matrix or, in some embodiments of the invention, on separate matrices per chain.

In some embodiments of the methods 200 and/or 300, it may desirable to ignore a pair of SNVs if the variant-to-variant distance between the two SNVs is smaller than a cutoff parameter. By appropriately selecting the cutoff parameter, specific distortions resulting from differences between sequencing technologies can be filtered out. In particular, the cutoff parameter may be 20. This optional filtering step is depicted in the method 200 by the block 217 and associated arrows, in dashed lines, and in the method 300 by the block 315 and associated arrows, in dashed lines. In embodiments, an additional cutoff parameter may filter distortions related to exceptionally large “gaps” as described previously.

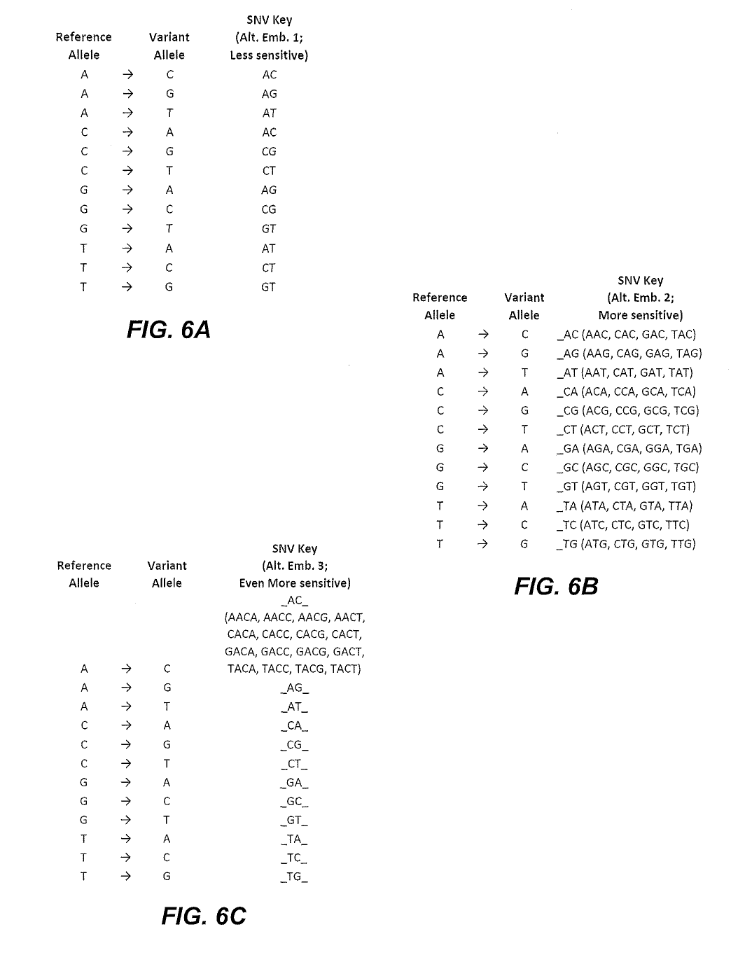

Variations on the concept of the SNV key described above (i.e., deviations from using the combination of the reference and variant alleles combined to form the SNV key) are possible, and allow the methods described herein to have increased or diminished sensitivity by, for example, increasing or decreasing the number of possible SNV keys and, accordingly, the number of possible pair keys. For instance, in embodiments, the SNV key is created without regard to which allele is the reference and which is the variant, as illustrated in FIG. 6A. That is, an SNV with a G reference allele and a C variant allele, results in the same pair key as an SNV with a C reference allele and a G variant allele (e.g., CG). In such embodiments, rather than 12 possible SNV keys, there exist 6 possible SNV keys. Correspondingly, there are 36 possible pair keys that could result in such embodiments, and the sensitivity (i.e., the ability to distinguish between genomes and/or determine degree of relatedness of the individuals associated with the genomes) of the distance modulo fingerprints may be decreased.

In other embodiments, it may be desirable to increase the sensitivity of the distance modulo fingerprints. One method by which this may be accomplished is to increase the number of possible SNV keys and, correspondingly, the number of pair keys that may result. For instance, the SNV pair key may be created not only from the reference and variant alleles (considered in order or not), but also from the nucleoside/base preceding the SNV, the base following the SNV, or both. That is, for a reference allele G and a variant allele A, the SNV key could be _GA, GA_, or _GA_, where the blank spaces represent the nucleoside preceding, the nucleoside following, or nucleosides both preceding and following the SNV. When the base preceding or following the SNV is included in the SNV key, 48 possible SNV keys result (as illustrated in FIG. 6B), while when both the bases preceding and following the SNV are used to generate the SNV key, there are 192 possible SNV keys (as partially illustrated in FIG. 6C). According to the results of the studies thus far, sufficient sensitivity may be achieved without requiring the implementation of these other embodiments.

The methods above result in a raw distance modulo fingerprint. The raw distance modulo fingerprints that result from the methods of FIGS. 4 and 5 have significant internal structure, both in the scale of the columns and the scale of the rows. The dimension (columns or rows, typically columns) that represents the distances between consecutive variants follows an exponential distribution and, accordingly, shorter distances between variants are more commonly observed than longer distances. This effect remains after “wrapping” the distribution (i.e., computing the reduced distances) using the modulo function. Further, if the cutoff parameter and the vector length parameter are different, there may be additional structure evident. The dimension (rows or columns, typically rows) that represents the combinations of variations (i.e., the pair keys), can each be a transition or a transversion. Because transitions are more common than transversions, SNV pair keys combining two transitions are more common than those combining a transition with a transversion and, in turn, these are more common than combinations of two transversions. All of the internal structural information is inherent to the method and does not add additional information about the genomes represented by the distance modulo fingerprints. Accordingly, it may be helpful to remove this non-informative structure by normalizing the distance modulo fingerprint to remove the internal structure.

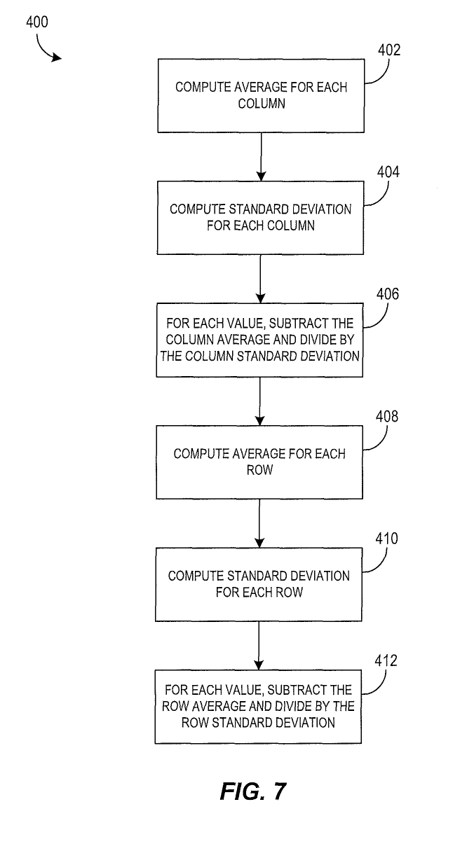

FIG. 7 depicts an example method 400 for normalizing a raw distance modulo fingerprint generated according to methods FIG. 4 or 5. The normalization method 400 involves computing the average and standard deviation for each column in the matrix (blocks 402 and 404, respectively). Thereafter, a Z-score is computed by subtracting the average value for each column from each value in the column, and dividing the standard deviation for each column into each value in the column (block 406). It should be understood that the Z-score (also known as a standard score) represents the signed number of standard deviations the value is above the mean. The method 400 also involves computing the average and standard deviation for each row in the matrix (blocks 408 and 410, respectively). Thereafter, the average value for each row is subtracted from each value in the row, and standard deviation for each row is divided into each value in the row (block 412).

It will be appreciated that additional utility may be obtained by adjusting fingerprints for population (e.g., ethnic or otherwise) to remove biases toward European (or other) populations that may be present in the reference genome(s) (e.g., the freeze or freezes from which initial representations are generated). For instance, the distance modulo fingerprints may be better sensitized to recognizing the relatedness of individuals if the distance modulo fingerprints are normalized to the population to which the individual(s) belong.

In principle, a “population” for purposes of adjusting or normalizing can be selected based on any selected trait or traits. In some variations, the population is selected based on a phenotypic trait, e.g., a disease condition or physical attribute. In some variations, the population is selected based on geographic origin, ethnicity, race, sex, or other criteria. If established scientific criteria do not exist for defining the population, then individuals can be classified by whether they self-identify as a member of the population, e.g., using a questionnaire.

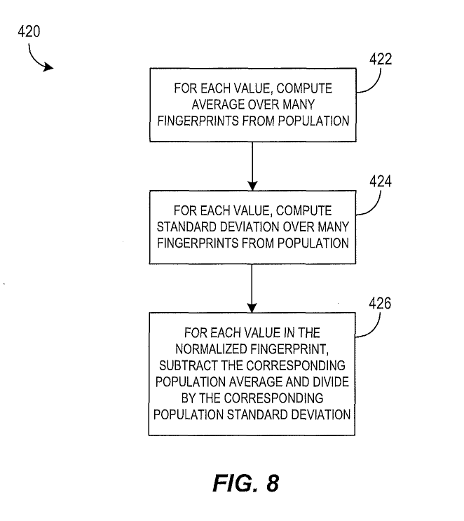

A method 420 for adjusting distance modulo fingerprints for population is depicted in FIG. 8. Generally speaking, the method 420 involves generating a population fingerprint for the population in question. The population fingerprint is actually two matrices—a first matrix comprising averages, and a second matrix comprising standard deviations. Thus, for each value in the distance modulo fingerprint, the average is computed over a set of many distance modulo fingerprints from the population in question (block 422) to generate a matrix of averages, and the standard deviation is computed over the same set of many distance modulo fingerprints (block 424) to generate a matrix of standard deviations. To perform population adjustment on a particular distance modulo fingerprint, then, each value in the DMF may be adjusted by subtracting from the value the corresponding average (taken from the matrix of population averages) and dividing it by the corresponding standard deviation (taken from the matrix of population standard deviations) (block 426). In an alternative embodiment (not shown), the computation at block 424 is not implemented such that no matrix of standard deviations is generated. In the alternative embodiment, method 420 is simplified, requiring only generation of the matrix of averages (block 422) and performing the population adjustment on a particular distance modulo fingerprint by adjusting each value in the DMF by subtracting from the value the corresponding average (taken from the matrix of population averages). Other alternative embodiments are further possible, including subtracting a matrix of medians (instead of a matrix of averages, as described above), subtracting a matrix of medians and dividing by a matrix of median absolute deviations (instead of a matrix of standard deviations, as discussed above). Moreover, in some embodiments, population adjustment is performed on a distance modulo fingerprint that has been previously normalized according to the method 400.

The distance modulo fingerprints may be readily compared with minimal computation requirements and, of course reduced memory requirements relative to complete genome sequences. The distance modulo fingerprints generated by the methods 200 and 300 will generate be represented and/or stored as matrices of values, each value representing the number of times a given pair key occurs with a specific reduced distance (i.e., the actual distance modulo the vector length). Accordingly, each matrix has dimensions dictated by the number of pair keys (e.g., 144 for the configuration depicted in FIG. 3) and the vector length (e.g., 20, 50, 100, etc.), and each value may be represented by an 8-bit, 16-bit, or 32-bit integer in the case of a raw DMF, or a floating point value in the case of the normalized and/or population-adjusted fingerprints. (Of course, there is no requirement that the values be represented by any specific number of bits, so long as the number of bits used is sufficient to represent the required values.)

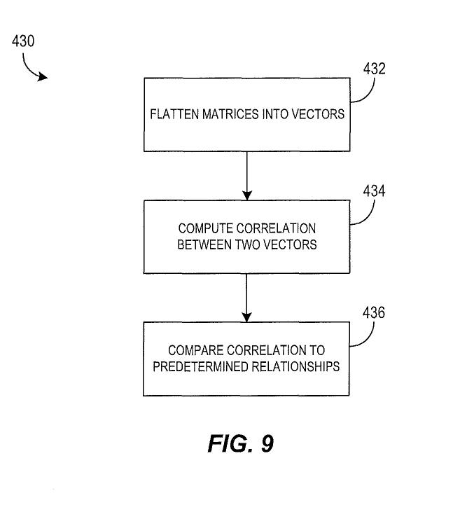

FIG. 9 depicts an example method 430 of comparing distance modulo fingerprints. Two distance modulo fingerprints may be compared to one another by first flattening the matrix representing each DMF to a vector (block 432). This may be accomplished, for example, simply by concatenating the rows of each of the matrix, such that each matrix is transformed into a corresponding vector. Computing a correlation—for example a Spearman correlation—between the two vectors (block 434) will allow the vectors and the corresponding genomes to be compared to determine one or more of a variety of characteristics, as described below, by comparing the correlation between the two vectors to various predetermined relationships (block 436). (Other types of correlations can also be used. Two such other correlations are the Pearson correlation and the Kendall correlation.)

Experimental data have yielded some correlation values that indicate the various predetermined relationships for the implementations tested. For instance, using the Spearman correlation, a correlation (i.e., a Spearman rho value) greater than 0.95 would indicate the two DMFs represent the same genome sequenced with the same technology; a correlation around 0.8 would indicate the two DMFs represent the same genome sequenced with different technologies; a correlation around 0.5 would indicate the two DMFs represent the genomes of siblings; a correlation around 0.25 would indicate the two DMFs represent the genomes of a parent and a child; a correlation around 0.15 would indicate the two DMFs represent the genomes or more distant relatives; a correlation around 0.0 would indicate the two DMFs represent the genomes of unrelated individuals; etc. Of course, the specific predetermined correlation values or ranges of correlation values that point to particular familial relationships can be determined or refined for each implementation of the invention by comparing fingerprints for individuals of known familial relation, generated using the fingerprint implementation in question.

Of course, as alluded to above, the sensitivity of the methods and systems described herein, and the utility of the embodiments implementing different sensitivities, may be varied in a variety of ways. As described above, it is possible to adjust the sensitivity of the method and/or system by adjusting the number of SNV keys that are possible. However, the vector length parameter may also be varied to adjust the sensitivity of the method. For instance, distance modulo fingerprints generated using a vector length of 20 may perform quite well for determining close family relationships, but may or may not perform as well for population analyses. Population analyses may experience better performance from distance modulo fingerprints generated with a vector length of between 100 and 150 and, specifically, with a vector length of 120.

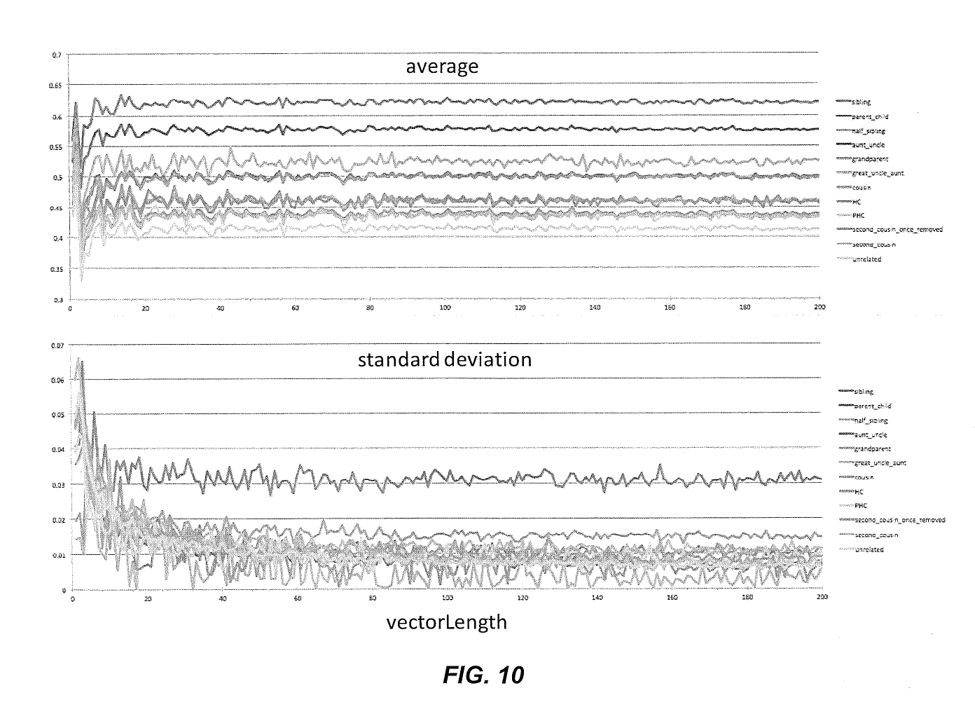

FIG. 10 depicts the results of a study evaluating the strength of each vector length value and, in particular, a graph of vector length versus average and standard deviation for various levels of relatedness in a large family pedigree, from siblings to second cousins and unrelated individuals. It will be understood that the data in FIG. 10 are not intended to be limiting, as the data are generated using fingerprints generated from a particular set of genome data, according to a specific set of parameters. As described throughout this specification, the parameters, genome data, and other aspects of the fingerprints may vary. In any event, the observed results of the study, depicted in FIG. 10, show that as the vector length parameter grows, the typical correlation observed for each relationship distance converges to a characteristic value. For some relationships, the value converges rather quickly. For example, for certain relationships, a vector length as short as 20 is highly correlated with very long vector lengths (in the range of 190 to 200). Accordingly, a vector length of 20 may be useful for determining some relationships, including some close relationships (e.g., sibling, parent/child, half sibling, etc.). Shorter vector lengths result in lower memory requirements, faster generation of the distance modulo fingerprints, and faster comparison of distance modulo fingerprints.

In some embodiments, a minimal distance modulo fingerprint (also referred to herein as a “binary distance modulo fingerprint,” a “binary DMF,” and/or a “minimal DMF”) may be implemented. A binary DMF may perform quite well in some circumstances, especially circumstances such as determining whether one genome is the same as another. For example, when determining whether a specific genome that one is considering adding to a set is already part of the set, it may be especially useful to implement the binary DMF, as a binary DMF, due to its small size, will facilitate faster determination of whether the genome is already part of the set.

In one study, approximately 2,500 genomes were compared using binary DMFs. That is, a binary DMF was generated for each of the 2,500 genomes, and each genome was compared against every other genome in the set. The comparison was completed on a single processor with non-optimized code, and yet the comparisons—3,133,756 in all—were completed in just over a minute. In another study, approximately 6,300 genomes were compared using binary DMFs. A binary DMF was generated for each of the 6,300 genomes, and each genome was compared against every other genome in the set. The resulting 19,860,753 comparisons were completed in just less than nine minutes. Of course, the time required to process the comparisons could be further reduced by using optimized software and parallelized processing.

In general, the binary DMF is generated in much the same way as the DMFs described above, using a vector length of 2 and, therefore, yielding a matrix 144×2. However, for each pair key in the matrix, the analysis considers whether more of the reduced distances are 0 or 1 (i.e., whether there are more even or odd distances), and sets a bit to 0 if there are more even distances for the pair key and 1 otherwise (or vice versa).

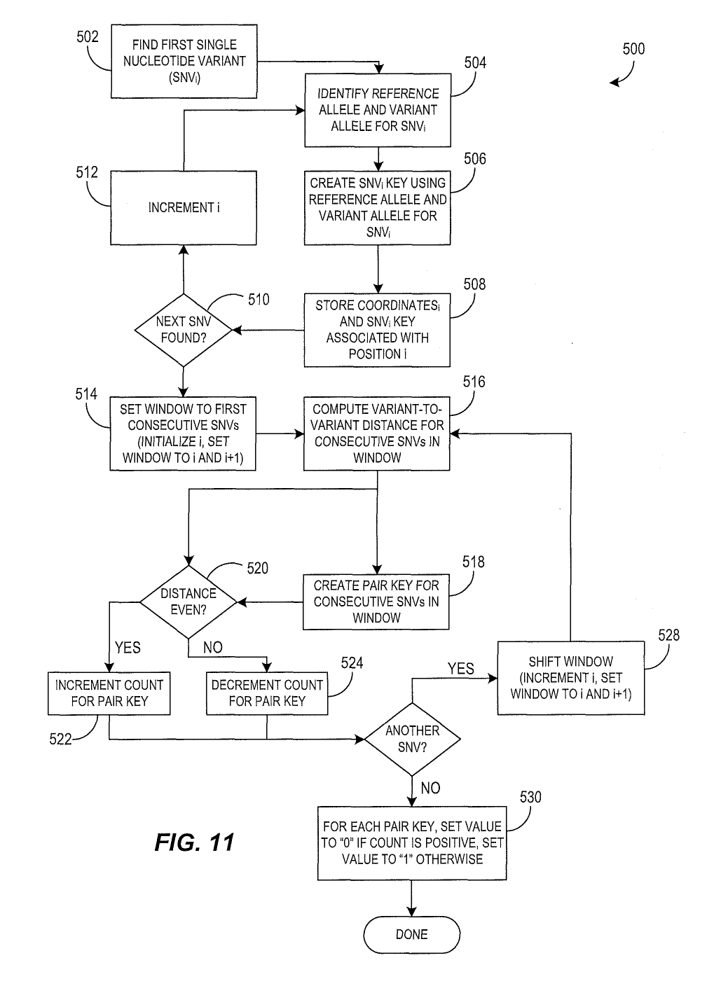

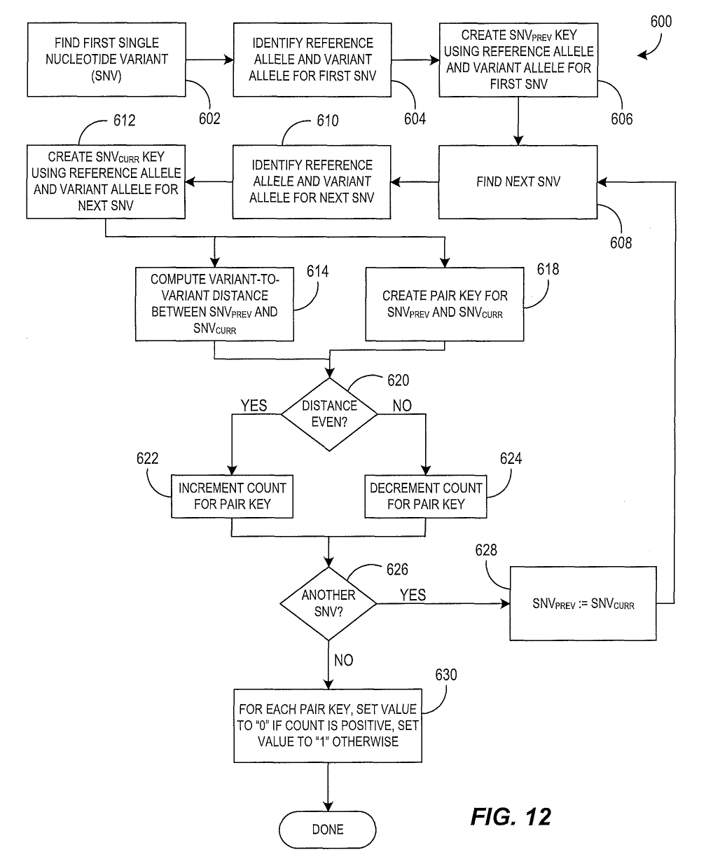

While the methods 200 and 300 can easily be adapted to generate the binary DMF as described above, FIGS. 11 and 12 depict two methods of generating the binary DMF. FIG. 11 depicts a method 500 for generating binary DMFs. The method 500 is similar in some respects to the method 200, with blocks 202, 204, 206, 208, 210, 212, 214, and 216 of the method 200 corresponding to blocks 502, 504, 506, 508, 510, 512, 514, and 516 of the method 500 and, accordingly, these blocks will not be described again with reference to FIG. 11. In the method 500, after computing the variant-to-variant distance for consecutive SNVs in the window (block 516), the method creates a pair key for the consecutive SNVs in the window (block 518) and determines whether the distance between the variants in the SNVs is an even number of bases (block 520). If the distance is an even number, the count for the pair key is incremented (block 522) and, if not, then the count for the pair key is decremented (block 524). If another SNV exists (block 526), the window is shifted (i is incremented and the window again set to SNVᵢ and SNVᵢ₊₁) (block 528), and blocks 516, 518, 520, 522, 524, and 526 are repeated. If no more SNVs exist, the value for each pair key is set to 0 if the count is positive, or 1 otherwise (i.e., if the count is negative or zero) (block 530), and the method 500 of determining the binary digital genomic fingerprint for the set of data is complete.

Of course, if one accounts for the asymmetry caused by setting the value to 1 when the count is negative or zero, even and odd can be exchanged without affecting the outcome of the method—that is, the method 500 could increment the count for the pair key at block 522 if the distance is not even at block 520, and could decrement the count for the pair key at block 524 if the distance is even at block 520. Similarly, at block 528, the value for each pair key may be set to 0 if the count is negative, and set to 1 otherwise. Additionally, though not depicted, the method 500 may also include the distance filter (depicted by block 217 in the method 200) that removes specific distortions resulting from differences between sequencing technologies.

FIG. 12 depicts an alternate method 600 for generating binary DMFs. The method 600 is similar in some respects to the method 300, with blocks 302, 304, 306, 308, 310, 312, 314, and 318 of the method 300 corresponding to blocks 602, 604, 606, 608, 610, 612, 614, and 618 of the method 600 and, accordingly, these blocks will not be described again with reference to FIG. 12. In the method 600, after computing the variant-to-variant distance between SNV_{PREV} and SNV_{CURR} (block 614), and creating the pair key for the consecutive SNVs SNV_{PREV} and SNV_{CURR} (block 618), the distance between the variants SNV_{PREV} and SNV_{CURR} is evaluated to determine whether it is an even number of bases (block 620). If the distance is an even number, the count for the pair key is incremented (block 622) and, if not, then the count for the pair key is decremented (block 624). If another SNV exists (block 626), the data for SNV_{PREV} are set equal to the data for SNV_{CURR} (block 628), and blocks 608, 610, 612, 614, 618, 620, 622, 624, and 626 are repeated. If no more SNVs exist, the value for each pair key is set to 0 if the count is positive, or 1 otherwise (i.e., if the count is negative or zero) (block 630), and the method 600 of determining the binary digital genomic fingerprint for the set of data is complete.

Of course, even and odd can be exchanged without affecting the outcome of the method (taking into account any asymmetries, as described above)—that is, the method 600 could increment the count for the pair key at block 622 if the distance is not even at block 620, and could decrement the count for the pair key at block 624 if the distance is even at block 620. Similarly, at block 630, the value for each pair key may be set to 0 if the count is negative, and set to 1 otherwise. Additionally, though not depicted, the method 600 may also include the distance filter (depicted by block 315 in the method 300) that removes specific distortions resulting from differences between sequencing technologies.

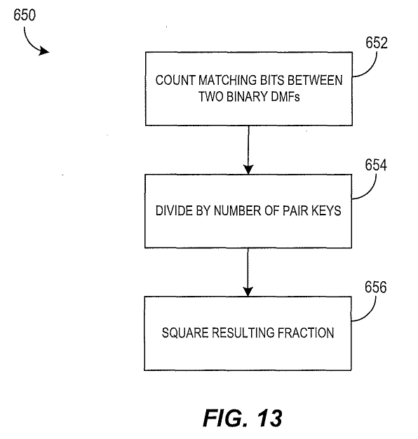

The binary DMFs may be compared in a variety of ways but, in particular, may be compared using a method 650 depicted in FIG. 13. To compare two binary DMFs, one need only count the number of pair keys that have the same value (i.e., either 0 or 1; that is, count the number of matching bits) (block 652), divide the number of matching bits by the number of pair keys (i.e., the number of bits) (block 654), and square the resulting fraction (block 656).

Of course any and/or all of the methods described above, including the methods 200, 300, 400, 420, 430, 500, 600, and/or 650, may be executed by systems comprising a computer (e.g., the computer 100) that may or may not be communicatively coupled to a network (e.g., the network 118) and/or to other servers (e.g., the server 120) and/or databases (e.g., the database 122). The methods 200, 300, 400, 420, 430, 500, 600, and/or 650 may be embodied as one or more applications, routines and/or modules stored on tangible, non-transitory, computer-readable media (e.g., the memory 110) such that a processor (e.g., the processor 108) may retrieve the instructions for execution. The instructions may be embodied and/or stored as one or more modules, routines.

In various embodiments, databases and related computer-implemented tools, such as online websites and webpages, may be created and implemented to store and provide access to genome fingerprints. In some embodiments, the database may be private, for example, accessible to only those with specific security permissions. In other embodiments, the database may be made public, for example, accessible to anyone. In some embodiments, the database may be implemented as one or more online databases accessible via a computer network, for example, database 122 associated with server 120 and accessible via network 118, as shown in FIG. 3.

Need for such database-centric solutions arises as the number of known genomes expands, such that genomic management, identification, and analysis has become drastically more complex. In some embodiments, a database of genome fingerprints may be used to determine which individuals have been recruited in multiple studies or to find cryptic relatedness in study populations that will cause statistical issues. In other aspects a fingerprint based database may be used to provide answers to common genome analysis questions, including, for example, determining whether a certain genome has been seen before; whether similar genomes have been seen before; whether genomes of relatives have been seen; or what genome or genomes are most similar, at least with respect to those genomes stored in the database.

The database may be part of a fingerprint management system. The use of the management system, for example, could allow researchers to manage data from large numbers of genomes through fingerprints. For example, a public database of genome fingerprints can support several applications (e.g., study design “matchmaking”), while maintaining privacy. In another aspect, the database may store and provide a method for computing personalized allele frequencies without requiring prior knowledge of populations.

In other aspects, the fingerprint management system may provide open source tools for implementing local, private fingerprint databases. In such an aspect, researchers installing a local copy of the management system are able to directly use genome fingerprinting in their research.

In other aspects, a public database of genome fingerprints may be used, the public databases using an authorization and authentication model to mitigate privacy concerns, but at the same time making all fingerprints available to facilitate creating and study populations easier, population identification faster, and to allow more collaboration in the research community via “data matchmaking.”

In other aspects, the accumulation of known genomes (with associated fingerprints) in databases allows analyses not previously possible. In particular, the combination of the public genome fingerprint database with large databases of known genomes like Kaviar, as described in [CITE Glusman 2011], which is incorporated by reference herein, enables the computation of precise, personalized allele frequencies and genotype frequencies.

As described above, in certain embodiments, computer-implemented tools are disclosed for creating private fingerprint databases. For example, the fingerprint management system, as described herein, can allow for organization of fingerprints for creating fingerprints of various sizes and normalization levels, quickly querying those fingerprints, and running analyses on subsets of fingerprints.

In one embodiment, the fingerprint management system may be an executable file or set of files, program or programs, or code able to be installed and used on a variety of computing operating systems (e.g., Linux systems, Microsoft systems, Apple systems, etc.).

In other aspects, the files or code may be open source code made available from a public repository under a particular code library.

In other aspects, the fingerprint system may support the indexing of multiple sizes of fingerprints and different normalization versions to support the development of algorithms and data exploration, to offer multi genomic analysis results, and provide visualizations of collections of fingerprint data.

For example, a specific online embodiment may include creating an Amazon Web Services (AWS) Lambda function (aws.amazon.com/lambda) as a NodeJS (e.g., a specific JavaScript runtime environment) deployment package that can be used to easily translate genomic source data into fingerprints that are stored on the researcher's Amazon S3 AWS account. In such an implementation, the fingerprint database system may use a modular architecture based on microservices, as described in [CITE Bahsoon 2016], which is incorporated by reference herein.

In the specific embodiment, the database may be built using, for example, the “MEAN” software stack (MongoDB, Express, Angular2, NodeJS) with frontend visualizations using D3 (d3js.org) and a REST (Representational state transfer) API backend as a scalable high availability web service.

The MongoDB (i.e., a NoSQL based database implementation) may be used to store and support expansion to hundreds of thousands of genome fingerprints. To support scaling to millions of genomes, alternative solutions may be used, including in-memory data stores like (e.g., Redis (redis.io)) and distributed graph databases such as Titan (titan.thinkaurelius.com).

In various embodiments, as described herein, a public genomic fingerprint database may be created. In some aspects, the public fingerprint database may facilitate creation of study populations, genomic analysis, and matchmaking between researchers. However, such public availability of fingerprint information may raise significant privacy concerns, e.g., metadata about particular fingerprints could be used to create likely matches to clinical data already possessed by a researcher. Accordingly, as described further herein, in one embodiment, a public genome fingerprint database may be characterized and add data in three stages: Public Data, Private Data, and Federation, with each data level designating a particular privacy or security level.

In Stage 1, the genome fingerprint database includes only fingerprints computed from Public Data, defined as sets of genomes that any qualified individual can obtain freely for research purposes.

In Stage 2, the database also includes fingerprints computed from Private Data as submitted by researchers. The privacy requirements for the private data fingerprints may be defined, such that addition of the fingerprints to the database required the fingerprints to meet a specific level of privacy or authorization.

In some aspects, data access to the database is granular, with each attribute of a resource and its metadata having individual permissions or residing as part of a group policy. Community researchers who submit fingerprints to the database are able to select an authorization level for their data and provide their contact information and select from several methods for requesting data access. The private fingerprint database may use data authentication and authorization to protect the system and keep the information private.

In a specific embodiment, use of a public identity provider, such as provided by Google, Amazon, or Auth0, allows users to create accounts to access the private data available on the fingerprint server. Such a system may be modeled around the Amazon Identity Access and Management (IAM) system, with users able to be assigned to groups and assume roles with specific permissions.

In certain aspects, different data authorization categories may be offered, e.g.: Public, Institution, Registered, and Private. Public authorization requires login with a public identity provider only. Institution authorization requires login with a specific institution's identity provider. Registered authorization requires login with an identity provider and a registered access attestation. Private authorization means that the user will receive information that there is a match in the database and the fingerprint identifier, but no access to the fingerprint and contact information for a researcher depending on the method selected by that researcher.

In some aspects, a user of the database system may select methods of contact. For example, a user may select the following methods to be contacted by another user: Website, Email, Phone, and Anonymous Message. In other aspects, the contact may be used to approve access requests. For example, once a user is contacted, the user can approve a request by another user by adding specific permissions for the other user or by adding the other user to a group or broader security policy.

In other aspects, and at the highest level of data restriction, a particular user may receive information informing that a match (within a specified threshold) has been found. The user may then send an anonymous message to the owner or researcher associated with the data, requesting more information. For this purpose, such private data may be stored on an encrypted microservice that may use policies or certificates to determine authorization for retrieval of matches and creation of contact requests.

In Stage 3, the database may have a Federation model that supports distributed queries into fingerprint databases stored at other institutions. The Federation model may allow sharing fingerprint databases and related data. For example, the Federation model allows fingerprint databases to communicate with each other so that a query to any connected fingerprint database can return results from all connected fingerprint databases based on the level of sharing selected.

In some embodiments, sharing modes are implemented. For example, Basic sharing mode allows requests that can return a yes/no result, Similarity sharing mode can return the fingerprint identifier and similarity match, and Full sharing mode can return the fingerprint identifier, similarity match, and fingerprint of specified size, subject to authorization and authentication restrictions, as described herein.

In other aspects, databases may store fingerprints to allow researchers or others to compute correlations between individuals with the goal of computing personalized allele frequencies, as described herein.

The methods and systems described herein have a number of advantages over prior methods and systems for performing analysis of genome sequences and genetic information. As already discussed, the methods and systems are agnostic to, and do not require knowledge of, the technology, reference, and encoding used to generate the genome sequence information, which means that the same methods can be used on databases containing sets of data generated using disparate technologies, references, and/or encoding schemes. Storage requirements for the data related to individual genomes is significantly reduced and, accordingly, large data sets require significantly smaller quantities of memory. Further, computation performed on the genome fingerprints is also faster (i.e., than other computations performed on the same processor) and requires significantly less memory.

Privacy is another benefit of the DMFs described herein. Because the DMFs retain only information about the frequency of various distances of SNVs relative to other SNVs, it is essentially impossible to reconstruct from a DMF the original genome representation, with shorter vector lengths being more effective for obscuring genetic sequence data and preventing reverse-engineering. As a result, it is difficult or impossible to identify or predict phenotypes associated with a particular DMF. Nor is it possible to reverse construct a set of genetic alleles to identify a specific individual from a DMF alone; such identification can only be made in the context of comparing a DMF to a DMF that has been previously prepared for the individual.

The DMFs described throughout this specification have a variety of uses including, by way of example and without limitation:

Simplifying the size and complexity of data required to uniquely characterize an individual's genome and differentiate it from genomes of other individuals of the same species;

Simplifying the size and complexity of data required to maintain library or database of individual genomes in a format that permits searching or querying or comparing, which has applications in all scientific and other fields (forensics; law enforcement) in which the maintenance and querying of a genome database for matches may be desirable;

Combining genome datasets more easily and in a manner that more readily facilitates identification and elimination of duplicate entries;

Establishing whether two genome representations are derived from the same individual—regardless of technology, genome freeze/reference, and/or encoding;

Establishing whether two genome representations are derived from closely related individuals;

Testing whether a new genome has already been observed (e.g., by comparing to a growing database of DMFs;

Querying a genome database to determine whether a query genome is present and/or whether a parent, sibling, grandparent, cousin, or other close relative's genome is present;

Testing for shared genomes in two or more studies;

Identifying population(s) of origin by comparing individual fingerprints to population fingerprints;

Selecting matched genomes by populations (e.g., finding most relevant control data, nearest neighbor search, etc.);

Computing kinship matrices from a collection of genomes, useful (in combination with sequence and phenotype information discernible from the original genome) for performing genome-wide association studies—removing a significant computational bottleneck;

Accelerating population structure studies by computing on a reduced representation of the genomes; and

Detecting gross chromosomal abnormalities by, for example, computing chromosome-specific DMFs.

### Variations on the Embodiments Described Above

Primarily, the embodiments contemplated above been described in a manner that takes advantage of the variant call format (VCF) files typically used to specify genetic information. In these files, as is known, one of a variety of reference genomes is compared against the genetic information of interest. Nucleotides that are the same as those of the selected reference genome are ignored. SNVs are denoted by, for example, the position at which the variation occurs, the reference allele, the variant allele(s), the genotype, and in some cases, a quality indicator.

Filtering Based on the Quality of the SNVs

In some embodiments, the methods for creating the DMFs may include one or more filtering steps to filter the DMFs to include only specific types of data. For example, a filtering step may remove or ignore SNVs that are below a pre-determined quality metric, which may be selected according to the standard used in a particular VCF file (or a particular set of data) and according to the amount of data that desired to be maintained in the DMF. Such a filtering step may occur, for example, between blocks 202 and 204 of the method 200, and/or between blocks 212 and 204 of the method 200, and/or between the blocks 302 and 304 of the method 300, and/or between the blocks 308 and 310 of the method 300, and/or between the blocks 502 and 504 of the method 500, and/or between the blocks 512 and 504 of the method 500, and/or between the blocks 602 and 604 of the method 600, and/or between the blocks 608 and 610 of the method 600. In any of these instances, if the next found SNV were below the selected quality threshold, the method would instead skip that SNV and find the next SNV.

Filtering Based on Zygosity

In embodiments, it may be advantageous to filter based on the zygosity of the variants. For instance, some embodiments will include heterozygous sites for the variant allele, while others will include homozygous sites for the variant allele. That is, for some variants specified in a VCF file, the genome in question will be homozygous at the site of the variation (i.e., both copies of the allele will be the same variant allele—for example, the reference could be G while both copies of the variant are A), while for other variants specified in a VCF file, the genome in question will be heterozygous at the site of the variation (i.e., the two copies of the allele will be different—for example, the reference could be G, one variant could be A and the other T, or one variant could be G and the other A, etc.). Filtering to use only heterozygous variant sites or only homozygous variant sites may be advantageous. For instance, by using only heterozygous sites, it may be possible to minimize reference biases. The use of heterozygous sites may also serve to reduce differences from individuals from different populations, and increase the difference between correlations of sibling pairs and correlations of parent to child, each of which may be desirable for certain analyses.

One disadvantage of using heterozygous sites only is that it reduces the number of SNVs available for the fingerprint and, therefore, reduces the resolution of the fingerprint. This, of course, is less of an issue for whole genome fingerprints than for chromosome, subchromosome and exome-based fingerprints.

Weighting Based on Zygosity

As alluded to above (and generally known), at any given diploid position in the genome, an individual can fall into any of four different categories:

0/0: homozygous reference (both alleles correspond to the reference allele)

0/1: heterozygous with one reference allele and one alternative allele

1/2: heterozygous with two alleles, neither of which matches the reference allele

1/1: homozygous for an alternative allele (both copies are the same, but different from the reference allele).

Diploid positions that are 0/0 are excluded by convention from the VCF files that typically specify genetic information.

As described above, in embodiments of the fingerprints described herein, the method considers SNVs regardless of whether they are homozygous (1/1) or heterozygous (0/1 or 1/2). In the embodiments described with reference to filtering based on zygosity, the method may consider SNVs only when they are homozygous (1/1) or only when they are heterozygous (0/1 or 1/2). In additional embodiments, the method may consider only SNVs that are 0/1 heterozygous, or only SNVs that are 1/2 heterozygous.

The differences in hetero- and homozygosity can also be exploited in other ways. For instance, in embodiments, double weight may be given to 1/1 homozygous sites by increasing by 2 (rather than 1) the value of the cell in the matrix corresponding to the pair key and the reduced distance. (That is, at blocks 222 or 320, for example, the count can be incremented by two, rather than one.) In another embodiment, SNV pairs in which one SNV is heterozygous and the other is 1/1 homozygous, me be given additional weight in the same manner (by increasing the count by double).

Fingerprints Using Different Genome Portions

As described above, the fingerprints may be computed based on different portions of the genome. Fingerprints may be computed based on the genetic information of a while genome or a partial genome. Such a partial genome may include a chromosome, a pair of chromosomes, and/or a combination of consecutive or non-consecutive chromosomes. The partial genome from which a fingerprint can be computed may also include sub-chromosomal regions. In embodiments, the fingerprints are computed from regions having between 10 kilobases (kb) and 100 megabases (Mb), from regions having between 10 kb and 10 Mb, from regions having between 10 kb and 1 Mb, from regions having between 10 kb and 500 kb, from regions having between 10 kb and 100 kb, from regions having between 100 kb and 100 Mb, from regions having between 100 kb and 10 Mb, from regions having between 100 kb and 1 Mb, from regions having between 1 Mb and 100 Mb, from regions having between 1 Mb and 10 Mb, from regions having between 10 Mb and 100 Mb, from regions having fewer than 500 Mb, fewer than 100 Mb, fewer than 50 Mb, fewer than 10 Mb, fewer than 5 Mb, fewer than 1 Mb, fewer than 500 kb, fewer than 100 kb, and/or from regions having more than 500 Mb, more than 100 Mb, more than 50 Mb, more than 10 Mb, more than 5 Mb, more than 1 Mb, more than 500 kb, or more than 100 kb.

Computing Fingerprints from Genomes Assembled without a Reference

Recently, methods have been and are being developed to identify variants in genomic data from de novo assembly of raw data. This new modality affords fully reference-free variant identification, and yields graphs of diploid sequences that express variants in heterozygous state. The variants cannot be directly compared to variants expressed relative to a reference sequence without further computation. However, the fingerprints described herein may avoid the additional computation by constructing the fingerprints directly from the graphs of diploid sequences.

In embodiments, the genomic fingerprints can be constructed using heterozygous sites, rather than variants relative to a reference. That is, instead of looking at variants from a reference, and creating SNV keys from the reference and variant alleles, the alternative embodiment may look at heterozygous sites within the genome (or portion of the genome) as reconstructed via de novo assembly, and may create SNV keys from the two alleles at the heterozygous site. The distances between consecutive pairs of heterozygous sites (rather than the distances between consecutive variants relative to a reference) may be used to compute the reduced distances.

Genome Fingerprints and Masking

In various embodiments, a binary fingerprint may be generated using an alternative encoding strategy. As described herein, certain embodiments can encode a genome fingerprint as a matrix of numbers. In a different embodiment, fingerprints are generated by encoding (“masking”) fingerprints to generate binary strings. One advantage of the masking encoding method is that it enables highly efficient bitwise comparisons, which can be orders of magnitude faster than computing correlations on matrices of numbers, as described for other fingerprint embodiment herein.

In another aspect, masked fingerprints may retain more information per genome than other fingerprints and methods as described herein (for example other binary fingerprints). In one embodiment, for example, a raw fingerprint is first created with an even-number vector length, e.g. 6. Then, a mask is chosen that assigns each of the six columns in the raw fingerprint to one of two classes (0 and 1). Examples of masks are 010101 (which yields the same as a typical binary fingerprint), 011001, 011100, etc. The number of possible masks is given by the equation:

$$\frac{{C}_{vectorLength / 2}^{vectorLength}}{2}$$

Thus, for the described embodiment, there are 10 different possible masks for vector length of 6. For each mask, total counts are compared (the same as for the case for a typical binary fingerprint) and a binary digit encoding (a single binary digit) is computed that is the result of the comparison. One or more masks are then computed per SNV pair key and all resulting bits are joined to form a binary string.

In accordance with the above, and in various aspects, a mask may be chosen for each pair key, where the mask assigns a class value to each counting value corresponding to both the pair key and the reduced distance. In some embodiments, the class value may be assigned a value of 0 or 1.

In some aspects, computing a digit encoding for a mask of a pair key may include applying, for each counting value of the pair key, the assigned class value to the counting value to generate a modified counting value and comparing each modified counting value to compute the digit encoding.

In some aspects, application of masks to a fingerprint may include choosing, for a pair key, a first mask and a second mask and computing a first digit encoding for the first mask and a second digit encoding for the second mask. A string value may be determined from the first digit encoding and second digit coding, where the string value is a concatenation of the first string value and the second string value.

In other aspects, the digit encoding is a binary digit encoding, but, the masking method is not limited to binary digits, as further described herein.

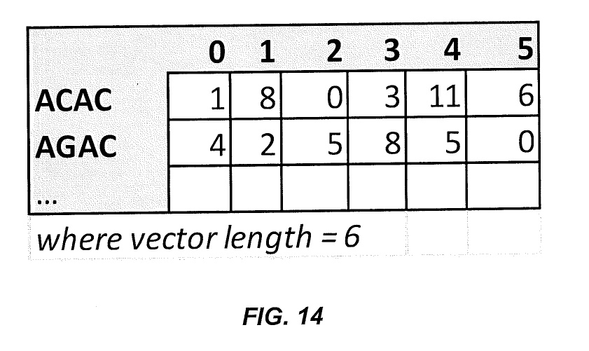

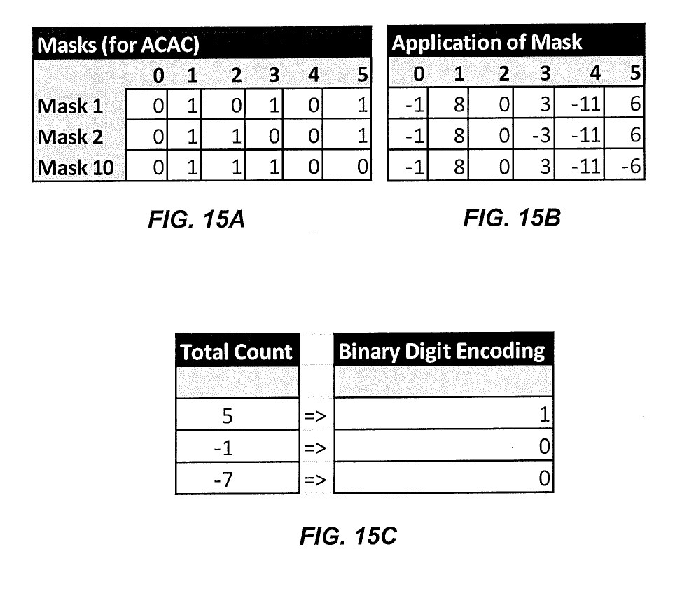

FIGS. 15A-15C relate to an embodiment and depict the application of binary masks to a genetic fingerprint to produce a mask-based binary string. Three masks, Mask 1, Mask 2 and Mask 10 are shown in FIG. 15A. Each mask has the same number of mask values as the vector length of a given fingerprint. For example, the masks of FIG. 15A have six mask values each corresponding to the vector length of six of the fingerprint shown for FIG. 14. The mask values determine specific classes, for example, as shown in FIG. 15A, classes 0 and 1 (as used for a binary mask).

Each mask may be applied to a pair key of a given fingerprint to compute a mask string. Because both the mask and the pair key row have the same length (a vector length of six, for FIG. 14), mask values are applied to the corresponding bins (cells) of the pair key. For example, FIG. 15B shows the masks of FIG. 15A applied to the pair key “ACAC” of fingerprint of FIG. 14. Because the masks of FIG. 15A are binary (having classes 0 and 1), application of mask values with class 0 to the pair key of the fingerprint cause the corresponding bin values of the fingerprint to become negative. In contrast, application of mask values with class 1 to the pair key of the fingerprint cause the corresponding bin values of the fingerprint to become positive. Thus, as shown in FIG. 15B, the fingerprint of bin values from FIG. 14 for pair key “ACAC” are shown, but with the mask values applied changing the “0” masked bins to negative values and the “1” masked bins to positive values. As shown, this is performed for each of the masks, Mask 1 to Mask 10. Other embodiments may use different class values (e.g., 0, 1, 2, 3, etc.). Still further, the different class values may take on different meaning for the mask values, e.g., instead of changing the corresponding fingerprint bins to negative or positive values, the mask values may indicate application of mathematical operations to the bin values of the fingerprint, such as doubling the value or applying some weight or percentage.

Next, as shown in FIG. 15C, for each mask, the sum of the computed negative and positive values is taken to produce a total count. If the total count value is positive or zero, then a binary digit encoding of “1” is produced. Otherwise, the value is “0.” In other embodiments, if the value is negative or zero, then a binary digit encoding of “0” is produced. Otherwise, the value is “1.” In either case, each of the binary digits may be stringed to together to form the binary string. The binary string may then be used for comparison purposes. In other embodiments, the mask string that is computed may reflect more information than provided in a binary string. For example, where the mask values have class values 0, 1, 2, a mask string may be computed that includes information based on the increased class values.

In an alternative embodiment, the SNV pair keys are not used and, instead, masks are computed on the combination of all SNV pairs, using larger values of vector length to achieve enough bits of information per genome. Due to the combinatorial nature of the method for generating possible masks, vector lengths of 6, 8, 10, 12, 14 and 16 can yield up to 10, 35, 126, 462, 1716 and 6435 bits, respectively. Thus, vector lengths of 12, 14 or 16 can be sufficient for producing enough bits of information per genome to support most applications. In some aspects, available genomes are used to train the system by choosing optimal sets of masks.

Genome Fingerprints from Genotype Data

Throughout this description, various embodiments have transformed variant data or data of heterozygous sites observed from whole-genome sequencing or exome sequencing according to the distances between pairs of variants or heterozygous sites. An alternative set of embodiments will be described below, which has both similarities and differences from the methods described above.

Specifically, it has historically been less expensive to use genotyping arrays to obtain genetic information on individual samples. For this reason, there are very large numbers of already genotyped samples, and most consumer applications involve genotyping arrays. Genotyping arrays include predetermined lists of specific variants to be tested; typical reports enumerate, for each variant tested, its single nucleotide polymorphism (SNP) identifier (known as an “rsid”), chromosomal location, and observed genotype.

Using these data, an alternate type of genomic fingerprint may be created. Instead of looking at variant pairs (or pairs from heterozygous sites), the modified method focuses on individual variants. For every reported variant, the key (similar to the SNV key) is the genotype. The resolution of the fingerprint can be adjusted, in one dimension, by changing the number of genotype keys. For instance, by counting genotypes GA and AG as different keys or the same key, or by including genotypes for nucleotide deletions. In an embodiment, the genotypes are alphabetically sorted, and the expected versus variant genotype is ignored, such that GA and AG are the same genotype. This arrangement yields 10 possible keys: AA, AC, AG, AT, CC, CG, CT, GG, CT, and TT.

Because the genotypes are considered individually, there are no associated distances between them, as would have been the case with the SNV keys described previously. Instead, the numerical portion of the rsid is used. While the numerical portion of the rsid has no intrinsic biological meaning, it is nevertheless a convenient way to distribute the data evenly in the fingerprint matrix. More importantly, while the specific number of the rsid is meaningless, rsids are largely stable as identifiers, which makes them a very suitable source of information for creating fingerprints.

Like in other embodiments, to transform the rsid numbers into a manageable size matrix, a vector length parameter is used as a modulus, resulting in a matrix that has a size in one dimension equal to the number of keys (10, for example), and a size in another dimension equal to the vector length (e.g., 100, 120, 20, etc.). The resulting matrix is then normalized and compared by Spearman correlation (or other comparison method) as for the distance-based fingerprints described previously.

Joining Genome Fingerprints to Increase Resolution

As should by now be apparent, fingerprints of different sizes can be computed from the whole genome or exome or any other subset of the genome, and the amount of information preserved in the fingerprints will vary according to the size of the subset of the genome included. The amount of information necessary to use a fingerprint for a given purpose may vary according to the purpose—a fingerprint of one size may be sufficient to determine whether two genomes are the same person or a different person, but insufficient to determine whether two genomes are from siblings or other relationships, for example.

Of course, fingerprints computed using different vector length parameters would not be compatible for comparison. Thus, it is preferable to find a way that fingerprints of desired resolution could be created while not forcing all analyses to use the highest-resolution fingerprints.

In embodiments, fingerprints created using different vector lengths can be combined to create fingerprints with higher resolution. Fingerprints of different vector lengths may include overlapping information and, accordingly, while such fingerprints may be combined, combining two fingerprints with different vector lengths may not always yield the resolution of a fingerprint having a resolution equal to the sum of the two vector lengths. (For instance, combining fingerprints with vector lengths 10 and 20 will not yield the same information as a vector length of 30.) When the vector lengths of two fingerprints are coprime, a combined fingerprint of the two fingerprints will carry more information than if the vector lengths are not coprime. Further, when the vector lengths used are all prime, each is guaranteed to carry different, non-overlapping information and, accordingly, they can be combined in any combination by concatenation of the matrices to create fingerprints of greater resolution. For example, if, for data of a given genome or exome, one computes fingerprints using vector lengths 7, 11, 13, 17, 19, 23, 29, and 31, they could be combined in any combination to yield a fingerprint having the resolution of the sum of the vector lengths used for the combined fingerprints: in this case a resolution of up to 150 (including 7+11=18, 7+13=20, 7+13+19=39, 29+31=60, etc.).

In embodiments, the joined fingerprints have already been normalized according to the procedures described herein.

### Additional Use Cases of Various Embodiments

As described above, fingerprints may be computed for portions of a genome including, for instance, for a chromosome. It is possible, using fingerprints computed as described herein, to determine from a fingerprint for a random chromosome, to which chromosome the fingerprint corresponds, if one has a copy of the same chromosome (from another individual) with which to compare. This is because the fingerprints computed from a single chromosome are highly comparable across individuals (i.e., chromosome 1 fingerprints from two individuals are highly correlated), while fingerprints from different chromosomes are not correlated, whether from the same individual or different individuals. The comparison could be performed against a fingerprint derived from a single instance of the chromosome (namely, from one individual) or against an averaged set of fingerprints from several individuals.

In the same manner, it is possible, using fingerprints computed as described herein, to determine from a fingerprint for a genome or exome, from which species the genome is derived, if there are corresponding fingerprints against which to compare. That is to say, two whole-genome fingerprints for the same species will exhibit a high correlation, while two whole-genome fingerprints for different species will not be correlated. The same is true for fingerprints for an exome or a chromosome; the exomes or chromosomes of different species will not exhibit a correlation, while exomes or chromosomes from similar species will be correlated.

Because each variant's contribution to a fingerprint is independent of the others, it is possible to create higher resolution fingerprints by using smaller regions of the genome (e.g., 10 Mb, 1 Mb, 100 kb, etc.). Different resolutions of fingerprints may be useful for additional analyses, including, for example, detection of chromosome-level aneuploidies, detection of sub-chromosomal aneuploidies, admixture mapping, mapping of de novo scaffolds to a reference, detection of segmental duplications, identification of paralogous regions of the genome, and others.

In some embodiments, it is possible to use characteristics of the fingerprints to support some data forensics analysis. For instance, while in some embodiments, it may be desirable to exclude SNV pairs that have a distance between them that is smaller than a predetermined cut-off (e.g., 20) value, in order to exclude effects caused by technology/reference differences. By separately studying SNV pairs with distances below the pre-determined cutoff (e.g., 20), those effects can be used to determine the technologies used to generate the genome data set. Various batch effects and filtering steps can also be identified by extracting such signals from the resulting fingerprints.

The fingerprints generated from the methods described herein may also be used for de novo computation of populations. As described herein, de novo computation of populations may also be performed without the use of fingerprints (e.g., via clustering from variant data, in particular ancestry-informative markers). In either event, and in one aspect, rather than collecting genomic data from individuals in a particular (and often ill-defined) population, based on geography or ancestry, such as “Europeans,” “Africans,” etc., as has been done previously, populations may be identified based on the genome fingerprints described herein. In another aspect, genome fingerprints may be analyzed using any of a variety of statistical analysis methods including, e.g., principal component analysis (PCA), multidimensional scaling (MDS), t-distributed stochastic neighbor embedding (t-SNE), or other methods of dimensionality reduction analysis. In an embodiment, PCA is used to determine the closest population to a particular genome (e.g., to determine which set of fingerprints is closest to a particular fingerprint). K-means clustering and Classification And Regression Trees (CART) methods can be used to cluster the PCA results.

Additionally, while population sets are typically determined by selecting an unbiased, formative subset of variants that relates individuals of a particular population, this is time and labor-intensive. By contrast, using PCA on fingerprint data facilitates the data reduction without the need for selecting the variants, and can be applied as soon as the genome is available. PCA applied to fingerprints of different vector lengths provided results highly correlated with results from PCA applied to variants, with convergence to the same principal component axes as either the number or variants or the vector length increased. In fact, for a sufficient amount of data in either form, correlation between corresponding principal components was >0.99 for the first 5 to the first 10 components.

Indexing Genome Fingerprints

Accessing, searching and comparing fingerprints may be accelerated by indexing the fingerprints prior to use. In general, use of fingerprints provides a very significant increase in comparison speed relative to standard methods, enabling very computationally demanding applications, e.g., all-against-all comparisons in large data sets of genomes to identify close and distant relatives. Such comparisons can be further enhanced via indexing, which can be beneficial, e.g., for large-scale fingerprint comparison tasks.

In various embodiments an empty index is first created in the shape of a matrix with the same dimensions as the fingerprints to be indexed. Second, for each fingerprint, the bins with large (absolute) values are selected that are expected to be the most unique among all fingerprints (i.e., minutiae). A reference pointing back to the fingerprint being indexed is then added to the index, and at each of the matrix coordinates of such extreme bins. Finally, to query the index, the lists of fingerprint ids referenced at the matrix positions where the query fingerprint has extreme values are selected and such lists are merged. The fingerprint(s) most frequently present in the merged list may then be prioritized in a search or comparison.

In certain aspects, parameters (e.g., the cutoff to consider a value “large”) are used to optimize the sensitivity and efficiency of the query, where, for example, low cutoffs may increase sensitivity at the expense of computation time, while larger cutoffs may incur in false negatives.

In other aspects, frequently related pairs may be co-indexed at different stringencies.

In other aspects, alternative acceleration strategies, e.g., based on known categories of genomes or based on classifying fingerprints by likely population of origin, as described herein, may also be used.

In various embodiments, a computer-implemented method of indexing genome fingerprints may include creating an index, where the index has a first dimension and a second dimension in common with an index fingerprint to be stored in the index. The first dimension and the second dimension may correspond to one or more bin values where the bin values are indicative of one or more respective reduced distances determined from corresponding one or more actual distances between one or more pairs of consecutive single nucleotide variants (SNVs) in a portion of a genome. One or more minutiae values may then be determined from the one or more bin values and selected for the index fingerprint. An index reference may be added to the index fingerprint index, where the reference indicates one or more locations of the one or more minutiae values.

In some embodiments, the minutiae values are significantly different from the one or more bin values such that the minutiae bins values have respective reduced distances greater than or equal to an absolute value of 3.

Other various embodiments may involve querying the index. Querying the index can include, for example, submitting a queried fingerprint to the index. The queried fingerprint can have one or more bin values corresponding to a first dimension and a second dimension, where the first dimension and the second dimension of the queried fingerprint correspond to the first dimension and the second dimension of the fingerprint index. The querying can further generate a prioritization value where the prioritization value is proportional with a count of the one or more references corresponding to the minutia values of the index fingerprint. A prioritization value can be computed for a plurality of fingerprints and then the various prioritization values (and their respective fingerprints) can be analyzed to prioritize a search or comparison of fingerprints in the index with respect to the queried fingerprint.

In various embodiments, haplotype-specific fingerprints are generated and applied to whole-genome phasing. As used herein the term “haplotype” refers to a group of alleles within an organism that was inherited together from a single parent. Phased sequencing, or “genome phasing,” may be used to identify alleles on maternal and paternal chromosomes. This is different from typical whole-genome sequencing, which generates a single consensus sequence without distinguishing between alleles on homologous chromosomes.

Haplotype-specific fingerprints may serve a variety of uses because DNA samples exhibit effects of many different natural mixture processes, for example:

(1) Blood samples, the most common source of sequenced human DNA, contain diploid cells with two haplotypes, one maternal and one paternal. Each parental haplotype in turn is an alternating pastiche of the haplotypes of two grandparents, which themselves were formed in the same manner in a previous generation. Thus, every human genomic DNA sequence is formed by mixture of pre-existing haplotypes, altered by a small number of mutations.

(2) Population structure is likewise the result of independent assortment and recombination of the haplotypes present in the separate mating pools of reproductively isolated populations.

(3) Forensic samples are also mixtures of haplotypes derived from different individuals, and in this context identifying the source individuals is of interest.

(4) Due to accelerated mutation in many cancers, the rapid and differential growth of cells in a tumor causes a tumor biopsy to contain a heterogeneous mixture of mutant copies of the individual's germline haplotypes, and the range and abundance of these mutant haplotypes in the sample are of medical interest.

Modeling the effects of these types of mixture on genome fingerprints allows measurement and use of the information the fingerprints carry about each contributing haplotype to applications in identifying source individuals, source populations, and the manner in which source haplotypes have been mixed in a fingerprinted sample.

Because an increasing number of genome sequences are being phased, either experimentally or bioinformatically for example, as described in [CITE Glusman 2014], which is incorporated by reference herein, or powered by large collections of observed haplotypes, for example as described in [CITE McCarthy 2016], which is incorporated by reference herein, or by new, single molecule sequencing technologies, in one embodiment, the disclosed genomic fingerprinting method (e.g., for use with diploid cells) may also be adapted to create fingerprints of single haplotypes on a chromosomal or subchromosomal scale.

For example, haplotype fingerprints may cover the same segment of the genome as diploid fingerprints, and may be compared to identify close relatives and distinguish populations.

In one aspect, a phased diploid genome could be fingerprinted as an unphased diploid genome (using the methods disclosed herein) and as a collection of single haplotype fingerprints. The different types of fingerprints may then be further compared to determine the accuracy for different use cases. For example, combining the diploid and haplotype fingerprint information across all chromosomes can provide additional accuracy, but at least as much accuracy as the diploid-based fingerprint alone. The haplotype fingerprints may also be used to determine the size of genomic regions that can be confidently discriminated (i.e., distinguished from one another).

In another embodiment, fingerprinting methods for whole-genome phasing may be generated. Haplotypes estimated from diploid samples may carry a risk of switching error, in which two loci are estimated to be adjacent in a single haplotype, but are actually from two different haplotypes, for example for as described in [CITE Glusman 2014], which is incorporated by reference herein. Even when chromosome haplotypes are properly phased, they may not be sorted into the maternal and paternal sets. While some phasing methods rely on trio data, and therefore include identification of the parent of origin of each haplotype, other phasing methods rely only on population data or on experimental procedures; in such cases, and in certain embodiments, whole-genome phasing can provide additional information about a diploid genome relevant to cis-effects such as imprinting and epigenetic effects on expression or compound heterozygosity.

Accordingly, in certain embodiments, fingerprints may be used to detect switching errors and for whole-genome phasing. For example, when the two parents have different ancestries, switching errors are detected by comparing chromosomal regions to representative (or average) fingerprints from each population. Whole chromosomes may also be sorted into maternal and paternal sets by likely population of origin.

In another aspect, when the two parents share ancestry, a more nuanced method may be applied, which uses a database of chromosomal haplotype fingerprints from known individuals. For example, a fingerprint database may be constructed from the haplotypes of the founders in a set of trio data, e.g., from public genome data, and from the recently published database haplotype reference consortium, for example, as referenced in [CITE McCarthy 2016], which is incorporated by reference herein. This method is based on the evolutionary similarity between two individuals as reflected on every chromosome; thus, haplotypes from the same parent show the same pattern of similarity in the database of known individuals, but haplotypes from different parents should show less similar patterns. This method may be used to group chromosomal haplotypes by parent of origin even when the parents are from the same source population. In another aspect, the method may also identify a statistical level of confidence associated with the grouping or identification.

In another aspect, a minimum span of chromosome sequence that must be represented in a fingerprint in order to confidently classify it by parent of origin may be determined.

In another aspect, incorrectly phased haplotype regions may be detected using the haploid fingerprints.

In another aspect, the disclosed fingerprinting methodology is based on information accumulated across a large region, which may provide a significant improvement in classification power over a population-based phasing strategy that relies strongly on local information.

In another embodiment, “population fingerprints” are developed that summarize observed populations. Individuals from the same population may share some evolutionary history, and therefore, may share some SNV pairs counted in computing genome fingerprints. Accordingly, fingerprints of a population may be summarized, both to estimate the “center” of the population's fingerprints and their variability around that center (population diversity). Such “population fingerprints” have a variety of uses, including population assignment for individuals.

For example, in one aspect, fingerprints having a particular length (e.g., a vector length of 120) may be computed for each population in a known data set (e.g., the 1000 Genomes data set). The computation may involve a mathematical function to determine a characteristic of a particular population (e.g., by simple averaging of the fingerprints of the genomes in each population). Then the correlation may be computed between a fingerprint of a query genome and for a fingerprint for each population. In some embodiments, the genome is assigned to the population with which it is most strongly correlated. Testing for this method (e.g., via cross-validation) yielded that the correct population is identified as the best match for 2047 of 2504 query genomes (82% of cases). Also, if the 2nd or 3rd best matches are accepted in addition to the best match, then the success rate increases to 96% and 98%, respectively.

In another aspect, data may be considered at the continental level (i.e., the “continental resolution”). Such data can include, for example, but not limited to, data regarding Africa, America, East Asia, Europe and South Asia. Use of fingerprints with continental data yields strong correlations, where, in one example, the best match was identified for all but 42 admixed American genomes.

In another embodiment, the value of traditional summarization methods of the center (mean, median) and scale (standard deviation, median absolute deviation) as means of representing the population as a whole may be used. A summarized center of fingerprints from a sample of individuals in a population may be referred to as a “population fingerprint” and the summarized scale of the same sample may be referred to as the “population fingerprint diversity.” Fingerprints may be compared to determine whether a particular fingerprint belongs to a particular population. Such comparison may include any of: a) using the (similarity) score of an individual genome fingerprint compared to the population fingerprint, or b) using the distance between the individual genome fingerprint and the population fingerprint, relative to the population fingerprint diversity.

In another embodiment, population-adjusted fingerprints for individual genomes may be developed. As described in other embodiments herein, two levels of fingerprints for an individual genome may be used, i.e., a “raw” fingerprint and an internally “normalized” fingerprint. In the population-adjusted fingerprints embodiment, a third level of “population adjusted” individual genome fingerprint may be computed by subtracting the closest average population fingerprint. This adjustment may eliminate the information common to the population, allowing close relationships within a population to be evaluated more precisely. Alternative mathematical methods of adjustment of individual fingerprints relative to the population fingerprints may also be used. In addition, a metric of population assignment confidence may also be applied, the metric based on the residual amount of population information after adjustment. Population-adjusted fingerprints may also be used for computing relationships among individuals, as described elsewhere herein.

In various embodiments, fingerprint designs are quantified based on the level of interpretability versus privacy of the fingerprints. That is, in some embodiments, genome fingerprints can retain interpretable information to allow a determination of the origin of the genome from which that fingerprint was computed and/or to be able to make predictions of disease risks, etc. But, in other embodiments, the opposite is desired, where fingerprints are developed to maintain privacy, and therefore, not allow (or diminish the ability) of the fingerprint to be interpretable.

Like any hashing approach, genomic fingerprinting is an extremely lossy form of compression of the input data. In one aspect, cryptographic hashing may retain the minimum possible information, ideally supporting no analysis of the output value beyond identity detection; a cryptographic hash creates identifiers suitable for “deidentifying” the data, and, thus maintaining a degree of privacy.

In another embodiment, the genome may be “compressed” by retaining only the SNVs that are currently known to be associated with a disease; this small fraction of the data, in some instances, can be the most sensitive information in the genome from a privacy perspective.

The fingerprints of various embodiments, as described herein, may, in some instances, be described as locality-sensitive hashes, where the fingerprints are data hashes of genomic information. This allows for encoding similar input data and similar output values, to provide a definition of similarity for, e.g., use in comparisons of the fingerprints. In certain embodiments herein, fingerprints may preserve evolutionary distances at both pedigree and population scales, and not specific variant values, thereby enabling analysis of relatedness and thus population structure, but not assessment of genetic disease risks, and therefore, in some instances, allow a degree of flexibility between privacy and interpretability.

In other aspects, fingerprints may provide information about degree of inbreeding.

In other embodiments, selecting an appropriate locality-sensitive hashing protocol, may be used to compute fingerprints that retain targeted functional information without exposing individual variant values, e.g., for providing a means of balancing the speed of large-scale analyses against data sharing and identifiability issues. Such hashing protocols may be considered as a basis for setting up or developing the systems used to store and access the fingerprints, e.g. a fingerprint database, as further described herein.

In one aspect, highly interpretable fingerprints are generated. For example, fingerprints may be generated to target specific kinds of information for retention, such as risk for a specific disease.

In one embodiment, a positive control is constructed for a “disease-specific fingerprint” containing allele values at a set of variants known to be relevant to a particular disease from known data (e.g., from genome-wide association studies (GWAS) studies). The control is then compared to “disease-targeting fingerprints” computed from subsets of variants near the genes containing the same disease-specific variants. In some aspects, the meaning of “near” can be varied (e.g., a mathematical value varied accordingly) to adjust the amount of data contributing to the fingerprint. Interpretability of the disease-specific and disease-targeting fingerprints, as well as untargeted genome fingerprints, can then be assessed as correlation with disease status on a set of genomes for which disease status is known.

In some embodiments, certain kinds of information may be retained in, or deduced from, the fingerprint (e.g., the degree of inbreeding associated with the genomic information of the fingerprint). In other aspects, factors and characteristics may be added to the fingerprint to improve the correlation with the targeted information. For example, including variants from additional gene or genes of interest may increase the correlation with disease status or disease risk. In other aspects, adjusting the fingerprint for population (as described elsewhere herein) may be used to increase or decrease the correlation. In other aspects, machine learning may be used to optimize the targeting parameters and to develop optimized fingerprints in a cross-validated, supervised learning setting.

In other aspects, functional information retained in genome fingerprints may be quantified. For example, to assess the level of privacy provided by genetic fingerprints, which may retain evolutionary distance information, fingerprints at various vector length values may be computed for control cohorts from specific disease studies. The fingerprints may be used to determine whether cases are distinguishable from controls based on fingerprints.

In other aspects, polygenic risk scores are computed for several specific diseases (e.g., from whole-genome data), where fingerprints may be used to predict the scores. The predictions may be tested in a leave-one-out cross-validation study of standard machine-learning classifiers, such as support vector machines (SVM), trained on the fingerprints of all but the test individual.

In other aspects, cryptographic hash “fingerprints,” which use random features to preserve as little information as possible, other than identity, provide a negative control at different values of the vector length of a fingerprint; any increase of prediction success over cryptographic hashing represents retention of information.

In another aspect, a different kind of assessment replaces increasing fractions of the genomic data with noise; this allows an estimate of the fraction of the input data that supports the retained information. Evolutionary distance information is supported by many independent variants; disease risk may be supported by a much smaller set of variants, or even a single variant. Such randomization allows for the distinguishing between information carried in small versus large numbers of variants, and therefore to determine whether a single variant's information can be recovered apart from its genomic context, representing a loss of privacy.

In various aspects, fingerprints may be optimized for privacy. As mentioned herein, a small set of individual SNVs have alleles known to be associated with specific diseases. One approach to improving the privacy of genome fingerprints is to explicitly exclude that set of SNVs (as well as any SNVs tightly linked to them) from the fingerprint computation. However, doing so requires the ability to identify these particular SNVs, which in turn requires information about how they are encoded relative to a specific reference genome.

In one aspect, when association with a phenotype is detectably retained by a specific definition of fingerprints, the features of a fingerprint that support the association may be characterized, and those features may be used to compute a residual fingerprint that specifically removes the detected association. For example, a principal components analysis (PCA) of the association can be used to provide a linear model of the association; subtracting the fingerprint predicted by the linear model provides a residual fingerprint that no longer contains the modeled association.

In some aspects, such model subtraction process may be used to remove the association regardless of the reference genome. Particular applications of the process include removing residual associates from inbreeding (as detected in fingerprints) or other instances where residual associations are detected, which provides the opportunity to enhance privacy for fingerprints in those situations as well.

In other embodiments, fingerprints may be used to perform kinship analysis and improve study designs. Such analysis may include, but is not limited to, large-scale relationship detection for computing large kinship matrices, identification of duplicate and related genomes across multiple data sets, evaluation of the population composition of data sets, and selection of matched controls for unbiased study design.

Knowledge of genetic relationships may be crucial to certain genetic studies, including analyses of disease heritability, linkage to genetic markers, and family-based association testing. Genetic information of related individuals may need to be removed from population-based association study cohorts to avoid bias. Existing methods for relationship detection and for computing kinship matrices require significant data preprocessing steps, including, for example: that the variants need to be expressed relative to the same reference; that different methods require different data formats, often requiring translation from one format to another; and that the representation of each variant needs to be “normalized” by selecting one of potentially many equivalent representation needs.

In certain embodiments, such preprocessing steps are not required before conversion to genome fingerprints, which are reference agnostic and easily computed from various formats. Because human choices are often required during preprocessing, minimizing preprocessing removes significant inefficiencies (in both time and manpower) from initial comparison-based genome analyses. Thus, the ability to very rapidly compare genomes using the fingerprints described herein can enable computations that were before too difficult or not scalable (e.g., computing large kinship matrices, choosing well-matched controls, etc.), enabling improved study designs.

In other aspects, personalized allele frequencies may be computed. For example, knowledge of allele frequencies may be crucial for filtering variants in certain disease studies. Population-specific allele frequencies may be more relevant to an individual than frequencies in the global population. For example, it is common practice to first identify the most likely population of origin of an individual, then use population-specific allele frequencies. However, there are two significant problems with this practice: (1) to date, few of the world's many ethnic populations have been genomically characterized, and, (2) an individual does not originate from a single “race”, but looking back k generations, is instead a mixture of up to 2—k source genomes, each of which might have contributions from uncharacterized ethnic backgrounds.

In contrast, for certain embodiments described herein, allele frequency computations are made tailored to each individual and that leverage the availability of thousands of complete genomes and related data from diverse populations (e.g., sources of Kaviar, as described in [CITE Glusman 2011], which is incorporated by reference herein, and are based on respective fingerprints (whole genome or per chromosome/region) computed from such data.

A specific embodiment may compare a query genome to each known population using fingerprints and use individual-to-population similarity scores to compute population-weighted allele frequencies.

In another aspect, a population-agnostic method may be used. In such an embodiment, a comparison is made, where a genome fingerprint is compared to a database of fingerprints such that the individual fingerprints in the database are ranked by similarity to compute a “nearest neighborhood” population for the query individual. In some aspects, the nearest neighbor genomes can be used as a reduced population for computing allele frequencies, bypassing the need for predefined populations.

In additional aspects, nearest neighbor genomes can be given equal weight or be weighted according to their similarity to the query genome. Parameters (e.g. similarity cutoff for neighborhood inclusion; weighting functions) may be used to estimate suitable allele frequencies and evaluate the accuracy of the predicted allele frequencies.

In certain embodiments, rapid estimation of pairwise degree of relationships and kinship matrices are enabled. For example, genome fingerprints can be used to estimate relationships very quickly, e.g., given two genomes, even in different representations and relative to different reference sequences, the genomes' respective fingerprints can be rapidly computed and comparison of such fingerprints can be nearly instantaneous. For example, the computational complexity of a single fingerprint comparison (Spearman correlation, O(m log m)) is a function of fingerprint size (m), not genome size (n; n>1000 m»m log m). For a population-scale cohort (N>100,000), an all-pairs comparison requires many comparisons (O(N̂2)), such that the speed of a single comparison may be a limiting factor.

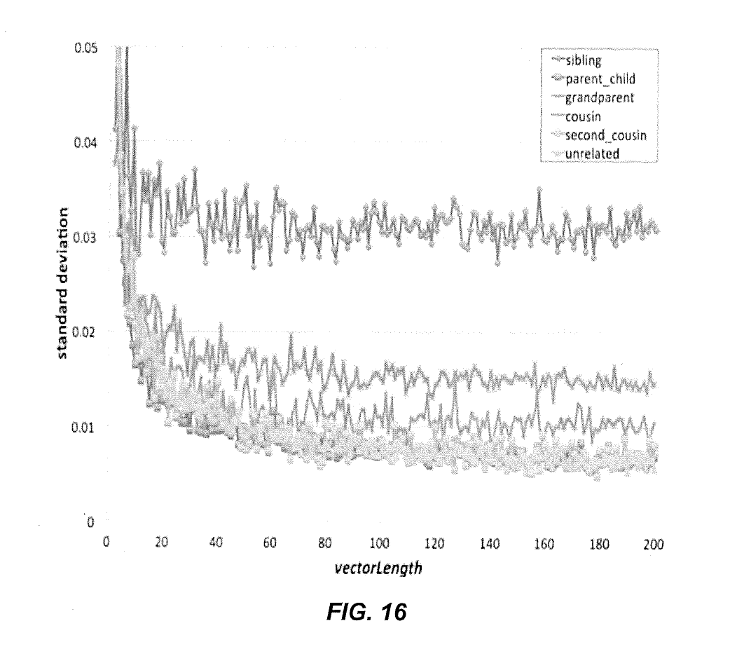

In some aspects, fingerprints, as disclosed herein, can distinguish close family relationships, e.g., up to second cousins (chance of sharing an allele by descent=1/32). Because prediction confidence improves with fingerprint length due to decreasing variance, particularly for unrelated pairs (FIG. 16), such relationships can be predicted using alternative methods for constructing fingerprints and different parameterizations, including using various values the vector length. For example, with reference to FIG. 16 and as depicted therein, the standard deviation (which may be used as a measure of error in some cases) for a parent-child comparison decreases from when the vector length is set a value below 20 to when the vector length is set to a value of 120. Similarly, FIG. 16 also shows that the respective standard deviation for other relationship types (e.g., cousins) likewise decreases when the vector length of the fingerprint is increased.

Other aspects may include the use of fingerprints computed from individual chromosomes and sub-chromosomal regions. In other aspects, the distribution of observed similarity values (ρ) as a function of vector length and the degree of relatedness in simulated and actual pedigrees from diverse populations may be used to estimate the degree of relatedness from ρ.

In other aspects, population adjusted fingerprints may enable higher resolution computation of relationships than normalized fingerprints.

In other aspects, fingerprint comparisons can also be used to give a very fast estimate of the coefficient of kinship (ϕ) between two genomes, and by extension, to quickly compute a kinship matrix even for large data sets. Kinship matrices may be approximated by standard linear mixed model approaches as described in [CITE Eu-Ahsunthornwattana 2014], which is incorporated by reference herein.

In other aspects, in addition to whole genome data, analogous systems and methods for comparison and kinship computation from exome sequencing data may be used, which may include different distributions of p than from the genome sequencing data.

In various embodiments, rapid identification of duplicate and related genomes may be implemented. This is because, for some instances, it is important to assess whether a set of genomes contains multiple genomes from a single individual, or whether any non-identical genomes are closely related.

In one aspect, fingerprints may be inputted for fingerprint-based similarity estimates. For example, fingerprints may be pre-classified by population and restricted based on close relationship to pairs in the same population, greatly reducing the number of comparisons. A faster method, for example, may use the locality preserved in each component of the fingerprint directly.

However, at some point any filtering method may lose sensitivity. Accordingly, in other aspects, approximate, pre-filtering methods may be used against rigorous methods that examine all pairs. For example, data may be combined for, e.g., meta-analyses or for other purposes, to detect whether certain genomes are present in more than one set, or whether the sets include closely related genomes. Accordingly, duplicate or related genomes may be identified in one set or two or more data sets. Such identification can lead to filtering the duplicate information.

In other aspects, different data sets may have batch-level differences, where such differences need to be estimated and accounted for in the comparison process. Such batch effects may be detected and removed to provide a further filtering effect.

In various embodiments, the disclosed fingerprints may enable quantitative assessment of population distributions. Common study design practice matches cases and controls based on a variety of variables thought to be potential confounders, typically including age, sex, ascertainment technology parameters, and population of origin (ancestry). Ancestry matching is particularly important and is typically done by identifying, for each case and each control, the population of origin relative to a small set of pre-established reference populations. In many cases, the granularity of the matching process can be as coarse as continent-level (African, European, East Asian, etc.). There are clear limitations, however, to such imprecision in matching, as population stratification is much richer than that simplistic model assumes. For example, individuals “from the same continent” may be very closely related or very distant. While this level of matching has been pragmatically appropriate to date, since the number of available controls has been small, future data, including fully sequenced genomes will count in the millions, enabling—for many types of analysis—much finer-grained matching of cases to controls. However, using current methods would result in a significant computational cost.

In aspects disclosed herein, the computation and comparison of fingerprints enable this quantitative assessment of population distributions to occur in reasonable time. In various aspects, fingerprints may enable continent and population level classifications, and also the distribution of pairwise similarities between genomes within each set. This enables precise evaluation of the contents of one set of genomes, and hence of the similarity of distribution of two or more sets of genomes.

In another aspect, large subsets of genomes may be selected from each set so as to maximize the similarity between the sets.

In another aspect, sets of genomes may be combined, minimizing redundancies and, where appropriate, genomes may be added from genomic databases, either public or private databases, as disclosed herein.

In other embodiments, genetic fingerprints may allow for precise selection of matched controls. That is, use of genome fingerprints enables the implementation of rational methods for precise selection of matched controls. In one aspect, given a set of potential control genomes, a selection of “ultimate matched controls” for a set of cases may be determined. For example, in one embodiment, for each case genome the closest matches in the set of possible controls may be found and ranked by similarity. Because such a computation may yield the same candidate control genome as ‘best match’ for more than one case genome, one of several possible procedures for assigning controls to cases may be used. For example, such procedures may involve: 1) accepting using the same matched control for more than one case, 2) applying a greedy algorithm to accept lower-ranked controls, 3) optimizing the selection to maximize the total similarity between the cases and controls, or 4) optimizing control assignment to achieve similar levels of similarity for all case/control pairs (i.e., minimize variance of pairwise correlations).

For some case genomes, it may be difficult or impossible to identify suitable controls under the above scenarios. Accordingly, in some aspects, the option of selecting automatically the subset of cases that have best controls above a matching threshold may be used.

The above matched control aspects may be used in conjunction with the online genomic databases, disclosed herein, to allow genomic study design to occur in a streamlined, precise and collaborative endeavor. For example, a researcher who just collected a set of case genomes could use an online database, as disclosed herein, to create a private database of their genomic fingerprints, evaluate the population distribution and privacy strength of the case genomes, query a public database to identify potential matched controls, and, based on the genome matching results, be advised to contact another researcher to establish a collaboration. Throughout this analysis and matchmaking process, no private genome information would need to be exposed.

FIG. 14 shows an example embodiment of a distance modulo fingerprint. As shown, the fingerprint of FIG. 14 has a vector length of 6, and, thus, six corresponding columns 0 to 5 (in the embodiment of FIG. 14, the columns, and not the rows, correspond to vector length). The rows of the FIG. 14 correspond to pair keys, including pair keys “ACAC” and “AGAC,” as shown. While only two pair keys are shown for FIG. 14, one or many pair keys may specified for any given fingerprint, for example, 144 pair keys may be specified for the fingerprint embodiment described with respect to FIG. 2. The cells (i.e., “bins”) of FIG. 14 show count values for each respective pair key with respect to each of the six possible modulus distances, where the count values indicate the number times a particular pair key was a modulus distance between consecutive SNVs. Thus, for example, and as shown in FIG. 14, the pair key “ACAC” shows that its respective SNV keys “AC” and “AC” were at a modulus distance of 4 for 11 times in the given genome or exome. For example, the SNV key “AC” could have been 40 base pairs away from the next SNV key “AC” yielding a remainder of 4 (because the vector length is 6); alternatively, the SNV key “AC” could have been 22 base pairs away from the next SNV key “AC” also yielding a remainder of 4. In both cases the count value at the bin (cell) with row “ACAC” and column 4 would be incremented. The other bin values of FIG. 14 would be incremented in a similar manner.

Throughout this specification, plural instances may implement components, operations, or structures described as a single instance. Although individual operations of one or more methods are illustrated and described as separate operations, one or more of the individual operations may be performed concurrently and, unless specifically described or otherwise logically required (e.g., a structure must be created before it can be used), nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

For example, the network 118 may include but is not limited to any combination of a LAN, a MAN, a WAN, a mobile, a wired or wireless network, a private network, or a virtual private network. Moreover, while only one computer 100 is illustrated in FIG. 3 to simplify and clarify the description, it is understood that any number of computers 100 are supported and can be in communication with the server or servers 120 and/or the database or databases 122.

Additionally, certain embodiments are described herein as including logic or a number of components, modules, routines, applications, or mechanisms. Applications or routines may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently or semi-permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions.

Similarly, the methods or routines described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs)).

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description should be read to include one or at least one and the singular also includes the plural unless it is obvious that it is meant otherwise.

Still further, the figures depict preferred embodiments of a system and methods for generating and comparing distance modulo fingerprints for purposes of illustration only. One skilled in the art will readily recognize from the preceding discussion that alternative embodiments of the structures and methods illustrated herein may be employed without departing from the principles described herein.

Upon reading this disclosure, those of skill in the art will appreciate still additional alternative structural and functional designs for a system and a process for generating and comparing distance modulo fingerprints through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The following list of aspects reflects a variety of the embodiments explicitly contemplated by the present application. Those of ordinary skill in the art will readily appreciate that the aspects below are neither limiting of the embodiments disclosed herein, nor exhaustive of all of the embodiments conceivable from the disclosure above, but are instead meant to be exemplary in nature.

1. A computer-implemented method of generating a representation of a genome, comprising: identifying for each single nucleotide variant (SNV) observed in a portion of the genome (i) a reference allele and (ii) a variant allele; joining the reference allele and the variant allele together to form a SNV key for each single nucleotide variant in the portion of the genome; and for each pair of consecutive SNVs: computing a variant-to-variant distance between the pair of consecutive SNVs; computing a reduced distance; creating a pair key; and incrementing a counting value corresponding to both the pair key and the reduced distance.

2. The computer-implemented method of claim 1, further comprising creating a matrix comprising one column for each pair key and one row for each reduced distance.

3. The computer-implemented method of claim 1, further comprising creating a matrix comprising one row for each pair key and one column for each reduced distance.

4. The computer-implemented method of any one of claims 1 to 3, wherein the portion of the genome is the whole genome.

5. The computer-implemented method of any one of claims 1 to 3, wherein the portion of the genome is a chromosome.

6. The computer-implemented method of any one of claims 1 to 3, wherein the portion of the genome is an exome, a transcriptome, or other set of the genome selected in a targeted way.

7. The computer-implemented method of any one of claims 1 to 3, wherein the portion of the genome is set of single nucleotide polymorphisms (SNPs).

8. The computer-implemented method of claim 7, wherein the set of SNPs is determined by a SNP chip analysis.

9. The computer-implemented method of any one of the preceding claims, wherein the variant-to-variant distance and the reduced distance are only computed, the pair key only created, and the value only incremented for pairs of consecutive SNVs on the same chromosome as each other.

10. The computer-implemented method of any one of the preceding claims, wherein the variant-to-variant distance is the absolute value of one less than the difference between the coordinates of the two SNVs.

11. The computer-implemented method of any one of the preceding claims, wherein computing a reduced distance comprises finding the remainder after division of the variant-to-variant distance by a vector length, n.

12. The computer-implemented method of claim 11, wherein the vector length, n, is 120.

13. The computer-implemented method of claim 11, wherein the vector length, n, is 20.

14. The computer-implemented method of claim 11, wherein the vector length, n, is 2.

15. The computer-implemented method of any one of the preceding claims, wherein creating a pair key comprises concatenating the SNV keys for each of the two consecutive SNVs.

16. The computer-implemented method of any one of the preceding claims, further comprising excluding variant-to-variant distances shorter than a pre-determined cutoff.

17. The computer-implemented method of any one of the preceding claims, further comprising: representing the genome as a matrix; and normalizing the matrix relative to a reference matrix derived from a set of genomes.

18. The computer-implemented method of claim 17, wherein normalizing the matrix relative to the reference matrix comprises: representing each genome of the set of genomes as a corresponding matrix; computing, for each position of the matrix, an average and a standard deviation for each matrix in the set of matrices from which the reference matrix is derived; and transforming the matrix by computing a Z-score for each value in the matrix, wherein the Z-score is the value, minus the average, divided by the standard deviation.

19. The computer-implemented method of either claim 17 or claim 18, wherein the set of genomes is a set of genomes from an identified population.

20. The computer-implemented method of any one of the preceding claims, further comprising: representing the genome as a matrix; and normalizing the matrix internally.

21. The computer-implemented method of claim 17, wherein normalizing the matrix internally comprises: computing a column average for each column in the matrix; computing a column standard deviation for each column in the matrix; for each value, subtracting the column average and dividing by the column standard deviation; computing a row average for each row in the matrix; computing a row standard deviation for each row in the matrix; and for each value, subtracting the row average and dividing by the row standard deviation.

22. A computer-implemented method of comparing genetic information, the method comprising: generating, from sequence data for a first genome, a first genetic fingerprint corresponding to the first genome; generating, from sequence data for a second genome, a second genetic fingerprint corresponding to the second genome; and determining a correlation between the first genetic fingerprint and the second genetic fingerprint, wherein each of the genetic fingerprints identifies, for each of a set of pairs of consecutive single nucleotide variants (SNVs) in the sequence data for the respective genome, a number of pairs of SNVs having each of a plurality of particular reduced distances.

23. The computer-implemented method of claim 22, wherein determining a correlation between the first genetic fingerprint and the second genetic fingerprint comprises determining a Spearman correlation coefficient.

24. The computer-implemented method of claim 22, wherein determining a correlation between the first genetic fingerprint and the second genetic fingerprint comprises determining a Pearson correlation coefficient.

25. The computer-implemented method of claim 23, further comprising comparing the Spearman correlation coefficient, p, to one or more thresholds to determine a relationship between respective samples from which the sequence data of the first and second genomes were obtained.

26. The computer-implemented method of claim 25, wherein the respective samples were from first and second human subjects, and wherein: p values of approximately 0.95 indicate the first and second human subjects are the same person; p values of approximately 0.8 indicate the first and second human subjects are the same person but that the technology used to obtain the sequence data for each is different; p values of approximately 0.5 indicate the first and second human subjects are related as siblings; p values of approximately 0.2 indicate the first and second human subjects are related as parent and child; and p values of approximately 0.15 indicate the first and second human subjects are family related other than as parent/child or siblings.

27. The computer-implemented method according to any of claims 22 to 26, wherein each of the genetic fingerprints is generated by: identifying for each SNV observed in the sequence data for the respective genome (i) a reference allele and (ii) a variant allele; joining the reference allele and the variant allele together to form a SNV key for each single nucleotide variant; and for each pair of consecutive SNVs: computing a variant-to-variant distance, the variant-to-variant distance between the pair of consecutive SNVs; computing a reduced distance; creating a pair key; and incrementing a counting value corresponding to both the pair key and the reduced distance.

28. The computer-implemented method of claim 27, further comprising creating a matrix comprising one column for each pair key and one row for each reduced distance.

29. The computer-implemented method of claim 27, further comprising creating a matrix comprising one row for each pair key and one column for each reduced distance.

30. The computer-implemented method of any one of claims 27 to 29, wherein the portion of the genome is the whole genome.

31. The computer-implemented method of any one of claims 27 to 29, wherein the portion of the genome is a chromosome.

32. The computer-implemented method of any one of claims 27 to 29, wherein the portion of the genome is an exome or other set of the genome selected in a targeted way.

33. The computer-implemented method of any one of claims 27 to 29, wherein the portion of the genome is a set of single nucleotide polymorphisms (SNPs) determined by a SNP chip.

34. The computer-implemented method of any one of claims 27 to 33, wherein the variant-to-variant distance and the reduced distance are only computed, the pair key only created, and the value only incremented for pairs of consecutive SNVs on the same chromosome as each other.

35. The computer-implemented method of any one of claims 27 to 34, wherein the variant-to-variant distance is the absolute value of one less than the difference between the coordinates of the two SNVs.

36. The computer-implemented method of any one of claims 27 to 35, wherein computing a reduced distance comprises finding the remainder after division of the variant-to-variant distance by a vector length, n.

37. The computer-implemented method of claim 36, wherein the vector length, n, is 120.

38. The computer-implemented method of claim 36, wherein the vector length, n, is 20.

39. The computer-implemented method of claim 36, wherein the vector length, n, is 2.

40. The computer-implemented method of any one of claims 27 to 39, wherein creating a pair key comprises concatenating the SNV keys for each of the two consecutive SNVs.

41. The computer-implemented method of any one of claims 27 to 40, further comprising excluding variant-to-variant distances shorter than a pre-determined cutoff.

42. The computer-implemented method of any one of claims 27 to 41, further comprising: representing the genome as a matrix; and normalizing the matrix relative to a reference matrix derived from a set of genomes.

43. The computer-implemented method of claim 42, wherein normalizing the matrix relative to the reference matrix comprises: representing each genome of the set of genomes as a corresponding matrix; computing, for each position of the matrix, an average and a standard deviation for each matrix in the set of matrices from which the reference matrix is derived; and transforming the matrix by computing a Z-score for each value in the matrix, wherein the Z-score is the value, minus the average, divided by the standard deviation.

44. The computer-implemented method of either claim 42 or claim 43, wherein the set of genomes is a set of genomes from an identified population.

45. The computer-implemented method of any one of claims 27 to 44, further comprising: representing the genome as a matrix; and normalizing the matrix internally.

46. The method of claim 45, wherein normalizing the matrix internally comprises: computing a column average for each column in the matrix; computing a column standard deviation for each column in the matrix; for each value, subtracting the column average and dividing by the column standard deviation; computing a row average for each row in the matrix; computing a row standard deviation for each row in the matrix; and for each value, subtracting the row average and dividing by the row standard deviation.

47. A scientific study comprising: providing an experimental group of organisms and a control group of organisms of the same species as the experimental group by: generating a representation of a genome for individual organisms according to the method of any one of claims 1 to 21; pairing organisms according to criteria that include a similarity between their respective genome representations; and assigning one member of a pair to the experimental group and another member of the pair to the control experimental group; applying an experimental variable to the experimental group of organisms; comparing one or more characteristics of the experimental group of organisms and control group of organisms after applying the experimental variable; and identifying a statistically significant difference between the experimental group of organisms and the control group of organisms for at least one of said characteristics.

48. The computer-implemented method of claim 1, wherein each of the single nucleotide variants is a heterozygous variant.

49. The computer-implemented method of claim 1, wherein the computing the reduced distance may comprise one or more of the following: scaling linearly, scaling using a nonlinear function, or binning.

50. The computer-implemented method of any one of the computer-implemented method claims, further comprising filtering the SNVs observed in the portion of the genome.

51. The computer-implemented method of claim 50, wherein the filtering comprises filtering the SNVs to consider only SNVs that are heterozygous.

52. The computer-implemented method of either claim 48 or claim 49, wherein the filtering comprises filtering the SNVs to consider variant quality.

53. The computer-implemented method of any one of claims 48 to 52, further comprising applying a weight value to the counting value.

54. The computer-implemented method of claim 53, wherein applying the weight value to the counting value comprises doubling the counting value.

55. The computer-implemented method of claim 53, wherein applying the weight value to the counting value comprises multiplying or adding the counting value with the weight value.

56. A method of identifying a characteristic of a set of genetic data, the method comprising: comparing a first representation of a portion of a first genome to a second representation of a portion of a second genome, wherein each of the first and second representations is generated according to the method of any one of the computer-implemented method claims, and wherein the characteristic of the portion of the first genome is known, and wherein the characteristic of the portion of the second genome is identified by its correlation to the portion of the first genome.

57. The method of 56, wherein the characteristic is the identity of a chromosome from which the genetic data were obtained.

58. The method of 56, wherein the characteristic is the identity of a species from which the genetic data were obtained.

59. The method of any one of the computer-implemented method claims, wherein the first representation is an average of a plurality of representations wherein the characteristic is shared.

60. The method of any one of the computer-implemented method claims, wherein the first representation is a single representation having the characteristic.

61. The method of any one of the computer-implemented method claims, wherein the portion of the genome has a length between 100 kb and 10 Mb.

62. The method of claim 61, wherein the representation of the genome contains sufficient data to perform one or more of detecting chromosomal aneuploidies and performing admixture mapping.

63. A computer-implemented method of generating a representation of a genome, comprising: identifying for each single nucleotide variant (SNV) observed in a portion of the genome (i) a first allele and (ii) a second allele, wherein the first allele and the second allele have a heterozygous relationship; joining the first allele and the second allele together to form a SNV key for each single nucleotide variant in the portion of the genome; and for each pair of consecutive SNVs: computing a variant-to-variant distance between the pair of consecutive SNVs; computing a reduced distance; creating a heterozygous pair key; and incrementing a counting value corresponding to both the pair key and the reduced distance.

64. A computer-implemented method of generating a representation of a genome, the method comprising: identifying in a portion of the genome heterozygous sites within the portion of the genome; cataloguing a location, a first allele, and a second allele for each of the heterozygous sites; joining the first allele and the second allele together to form an SNV key for each location of the heterozygous sites; and for each consecutive pair of heterozygous sites: computing a distance between the respective locations of the pair of heterozygous sites; computing a reduced distance; creating a pair key; and incrementing a counting value corresponding to both the pair key and the reduced distance.

65. The computer-implemented method of any one of the previous computer-implemented method claims, further comprising choosing a mask for each pair key, wherein the mask assigns a class value to each counting value corresponding to both the pair key and the reduced distance.

66. The computer-implemented method of claim 65, wherein the class value is one of the following values: 0 or 1.

67. The computer-implemented method of either claim 65 or claim 66, further comprising computing a digit encoding for a mask of a pair key, the computation comprising: applying, for each counting value of the pair key, the assigned class value to the counting value to generate a modified counting value; and comparing each modified counting value to compute the digit encoding.

68. The computer-implemented method of claim 67, wherein the digit encoding is a binary digit encoding and wherein the class value is one of the following values: 0 or 1.

69. The computer-implemented method of any one of claims 65 to 68, further comprising: choosing, for a pair key, a first mask and a second mask; computing a first digit encoding for the first mask; computing a second digit encoding for the second mask; and determining a string value from the first digit encoding and second digit coding, wherein the string value is a concatenation of the first string value and the second string value.

70. The computer-implemented method of claim 69, wherein the string value is a binary string value and wherein the class value is one of the values: 0 or 1.

71. A computer-implemented method of generating a representation of a genome, comprising: identifying, for each single nucleotide variant (SNV) observed in a portion of the genome, a variant allele; and for each pair of identified consecutive SNVs: computing a variant-to-variant distance between the pair of consecutive SNVs; computing a reduced distance; computing a contiguous sequence value; incrementing a counting value corresponding to both the contiguous sequence value and the reduced distance.

72. A computer-implemented method of generating a representation of a genome, the method comprising: identifying in a portion of the genome heterozygous sites within the portion of the genome; cataloguing a location for each of the heterozygous sites; for each consecutive pair of heterozygous site locations: computing a distance between the respective locations of the pair of heterozygous sites; computing a reduced distance; and incrementing a counting value corresponding to the reduced distance.

73. A computer-implemented method of generating a representation of a genome, the method comprising: identifying, for each single nucleotide variant (SNV) observed in a portion of the genome, a location of the SNV; and for each consecutive pair of SNV locations: computing a distance between the respective locations of the pair of SNVs; computing a reduced distance; and incrementing a counting value corresponding to the reduced distance.

74. The computer-implemented method of either claim 72 or claim 73, further comprising choosing a mask for each pair key, wherein the mask assigns a class value to each counting value corresponding to both the pair key and the reduced distance.

75. The computer-implemented method of claim 74, wherein the class value is one of the following values: 0 or 1.

76. The computer-implemented method of either claim 74 or claim 75, further comprising computing a digit encoding for a mask of a pair key, the computation comprising: applying, for each counting value of the pair key, the assigned class value to the counting value to generate a modified counting value; and comparing each modified counting value to compute the digit encoding.

77. The computer-implemented method of claim 76, wherein the digit encoding is a binary digit encoding and wherein the class value is one of the following values: 0 or 1.

78. The computer-implemented method of any one of claims 74 to 76, further comprising: choosing, for a pair key, a first mask and a second mask; computing a first digit encoding for the first mask; computing a second digit encoding for the second mask; and determining a string value from the first digit encoding and second digit coding, wherein the string value is a concatenation of the first string value and the second string value.

79. The computer-implemented method of claim 78, wherein the string value is a binary string value and wherein the class value is one of the values: 0 or 1.

80. A computer-implemented method of generating a representation of a genotype, comprising: identifying a plurality of single nucleotide polymorphisms (SNPs) in a portion of the genome, each of the plurality of SNPs having a corresponding numerical Reference SNP cluster ID (rsid) and a corresponding genotype; and for each SNP: computing a reduced value from the rsid; and incrementing a counting value corresponding to both the genotype and the reduced value.

81. The computer-implemented method of claim 80, wherein the computing reduced value from the rsid comprises computing the modulus of the rsid divided by a vector length.

82. A computer-implemented method of generating a representation of a portion of a genome, the method comprising: identifying a plurality of distance values in the portion of the genome; creating a first reduced representation of the portion of the genome by, for each of the distance values: computing a first reduced distance, wherein computing the first reduced distance comprises finding the remainder after division of the respective distance value by a first vector length, n1; and incrementing a counting value according to at least the first reduced distance; creating a second reduced representation of the portion of the genome by, for each of the distance values: computing a second reduced distance, wherein computing the second reduced distance comprises finding the remainder after division of the respective distance value by a second vector length, n2; and incrementing a counting value according to at least the second reduced distance; normalizing the first and second reduced representations of the portion of the genome to create, respectively, first and second normalized reduced representations; joining the first and second normalized reduced representations of the portion of the genome to create the representation of the portion of the genome.

83. The method of claim 82, wherein each of the distance values corresponds to the distance between a set of consecutive SNVs observed in the portion of the genome.

84. The method of either claim 82 or claim 83, wherein each of the distance values corresponds to the distance between consecutive locations exhibiting heterozygosity.

85. The method of any one of claims 82 to 84, further comprising: identifying a pair key associated with each of the plurality of distance values.

86. The method of claim 85, wherein identifying the pair key associated with each of the plurality of distance values comprises: identifying two single nucleotide variants (SNVs), the distance between the locations of the two SNVs defining the distance value; identifying for each of the two SNVs a reference allele and a variant allele; joining, for each of the two SNVs, the reference allele and the variant allele, to create an SNV key; joining the respective SNV keys created for each of the two SNVs to form the pair key.

87. The method of claim 86, further comprising incrementing each of the counting values according to the respective reduced distances and according to the pair key.

88. The method of claim 85, wherein identifying the pair key associated with each of the plurality of distance values comprises: identifying two heterozygous sites in the portion of the genome, the distance between the locations of the two heterozygous sites defining the distance value; identifying for each of the two heterozygous sites a first allele and a second allele; joining, for each of the two heterozygous sites, the first allele and the second allele, to create a key; joining the respective keys created for each of the two heterozygous sites to form the pair key.

89. The method of any one of claims 82 to 88, wherein n1 and n2 are both prime numbers.

90. The method of any one of claims 82 to 89, wherein n1 and n2 are co-prime.

91. The method of any of claims 82 to 90, wherein joining the first and second reduced representations of the portion of the genome to create the representation of the portion of the genome comprises concatenating the first and second reduced representations.

92. The computer-implemented method of any one of the computer-implemented method claims, further comprising identifying, based on the one or more of the variant-to-variant distances between the pair of consecutive SNVs or the reduced distance, one or more of the following: a commercial software technology used to generate a dataset associated with the portion of the genome, batch effects associated with the portion of the genome, post-processing functions associated with the representation of the genome, or filtering functions associated with the representation of the genome.

93. The computer-implemented method of claim 22 or any claim depending therefrom, wherein the sequence data for the first genome is one of the following: sequence data from a genome, sequence data from an exome, sequence data from a genotype array, or sequence data from a capture array.

94. The computer-implemented method of claim 22 or any claim depending therefrom, wherein the sequence data for the second genome is one of the following: sequence data from a genome, sequence data from an exome, sequence data from a genotype array, or sequence data from a capture array.

95. The computer-implemented method of claim 22 or any claim depending therefrom, wherein: the sequence data for the first genome is one of the following: sequence data from a genome, sequence data from an exome, sequence data from a genotype array, or sequence data from a capture array, and the sequence data for the second genome is a different one of the following from the sequence data for the first genome: sequence data from a genome, sequence data from an exome, sequence data from a genotype array, or sequence data from a capture array.

96. The computer-implemented method of claim 95, wherein sequence data for the first genome and the sequence data for the second genome come from the same individual.

97. A computer-implemented method of any of claims 17 to 19, further comprising augmenting the genome matrix to include one or more variances of a respective one or more individuals.

98. A computer-implemented method of claim 22 or any claim depending therefrom, wherein the first sequence data for the first genome includes genomic information associated with individuals indigenous to a particular geographic location.

99. A computer-implemented method of claim 22 or any claim depending therefrom, wherein the first and second genetic fingerprints are subjected to a dimensionality reduction analysis.

100. The computer-implemented method of claim 99, wherein the dimensionality reduction analysis is a principal components analysis (PCA), and wherein the PCA generates a set of PCA coordinates.

101. The computer-implemented method of claim 100, further comprising determining one or more clusters of related PCA coordinates based on one or more of the following clustering methods: k-means clustering or the Classification And Regression Trees (CART) method.

102. The computer implemented method of either claim 100 or claim 101, wherein the PCA is used to determine closest populations for one or both of the genetic fingerprints, irrespective of pre-defined populations.

103. A computer-implemented method of indexing genome fingerprints, comprising: creating an index, the index having a first dimension and a second dimension in common with an index fingerprint to be stored in the index, wherein the first dimension and the second dimension corresponds to one or more bin values, wherein the bin values are indicative of one or more respective reduced distances determined from corresponding one or more actual distances between one or more pairs of consecutive single nucleotide variants (SNVs) in a portion of a genome, wherein the index fingerprint has an identifier that identifies the fingerprint in the index; selecting, for the index fingerprint, one or more minutiae values determined from the one or more bin values; and adding to the index one or more references to the index fingerprint, wherein one or more locations of the one or more references correspond to the minutiae values of the index fingerprint.

104. The computer-implemented method of claim 103, wherein the minutiae values are significantly different from the one or more bin values such that the minutiae bins values have respective reduced distances greater than or equal to an absolute value of 3.

105. The computer-implemented method of either claim 103 or claim 104, further comprising querying the index, wherein the querying comprises: sending a queried fingerprint to the index, wherein the queried fingerprint has one or more minutiae values corresponding to a first dimension and a second dimension, wherein the first dimension and the second dimension of the queried fingerprint correspond to the first dimension and the second dimension of the indexed fingerprint; and generating a prioritization value, the prioritization value proportional with a count of the one or more references corresponding to the minutia values of the index fingerprint.

106. A computer-implemented method of adjusting distance modulo fingerprints for population, comprising: generating a statistics matrix including one or more statistics, the one or more statistics determined by taking statistical values in a set of distance modulo fingerprints (DMFs); and subtracting from each value in a particular DMF the one or more statistical values in the statistics matrix to determine a difference value corresponding to each value in the particular DMF.

107. The method of claim 106, wherein the one or more statistics can be one of the following: one or more averages, one or more medians or one or more modes.

108. A computer-implemented method of claim 106, further comprising: generating a deviations matrix including one or more deviations, the one or more deviations determined by taking the deviation with respect to the values in the set of DMFs, and wherein the one or more deviations in the divisions matrix correspond to the one or more statistics in the statistics matrix; and dividing the difference value corresponding to each value in the particular DMF by the corresponding one or more deviations in the deviations matrix.

109. The method of claim 108, wherein the one or more deviations can be one of the following: one or more standard deviations or one or more median absolute deviations.

110. The computer-implemented method of claim 22 or any claim depending therefrom, wherein one or more of the number of pairs of SNVs corresponding to the sequence data for the second genome is excluded from the sequence data for the second genome based on an exclusion factor.

111. The computer-implemented method of claim 22 or any claim depending therefrom, wherein one or more of the number of pairs of SNVs corresponding to the sequence data for the first genome is excluded from the sequence data for the first genome based on an exclusion factor.

112. The computer-implemented method of claim 110, wherein the exclusion factor is a probability for determining the likelihood that a particular SNV pair in the second sequence is excluded.

113. The computer-implemented method of claim 110, wherein the exclusion factor is an allowed minimal distance between the consecutive SNVs in the second sequence, wherein each SNV pair below the minimal distance in the second sequence is excluded.

114. The computer-implemented method of claim 110, wherein the exclusion factor is an allowed maximal distance between the consecutive SNVs in the second sequence, wherein each SNV pair above the maximal distance in the second sequence is excluded.

1. A computer-implemented method of generating a representation of a genome, comprising:
identifying for each single nucleotide variant (SNV) observed in a portion of the genome (i) a reference allele and (ii) a variant allele;
joining the reference allele and the variant allele together to form a SNV key for each single nucleotide variant in the portion of the genome; and
for each pair of consecutive SNVs:
computing a variant-to-variant distance between the pair of consecutive SNVs;
computing a reduced distance;
creating a pair key; and
incrementing a counting value corresponding to both the pair key and the reduced distance.

2. The computer-implemented method of claim 1, further comprising creating a matrix comprising one column for each pair key and one row for each reduced distance.

3. The computer-implemented method of claim 1, further comprising creating a matrix comprising one row for each pair key and one column for each reduced distance.

4. The computer-implemented method of claim 1, wherein the portion of the genome is the whole genome.

5. The computer-implemented method of claim 1, wherein the portion of the genome is a chromosome.

6. The computer-implemented method of claim 1, wherein the portion of the genome is an exome, a transcriptome, or other set of the genome selected in a targeted way.

7. The computer-implemented method of claim 1, wherein the portion of the genome is set of single nucleotide polymorphisms (SNPs).

8. The computer-implemented method of claim 7, wherein the set of SNPs is determined by a SNP chip analysis.

9-16. (canceled)

17. The computer-implemented method of claim 1, further comprising:
representing the genome as a matrix; and
normalizing the matrix relative to a reference matrix derived from a set of genomes.

18. The computer-implemented method of claim 17, wherein normalizing the matrix relative to the reference matrix comprises:
representing each genome of the set of genomes as a corresponding matrix;
computing, for each position of the matrix, an average and a standard deviation for each matrix in the set of matrices from which the reference matrix is derived; and
transforming the matrix by computing a Z-score for each value in the matrix, wherein the Z-score is the value, minus the average, divided by the standard deviation.

19. (canceled)

20. The computer-implemented method of claim 1, further comprising:
representing the genome as a matrix; and
normalizing the matrix internally.

21. The computer-implemented method of claim 20, wherein normalizing the matrix internally comprises:
computing a column average for each column in the matrix;
computing a column standard deviation for each column in the matrix;
for each value, subtracting the column average and dividing by the column standard deviation;
computing a row average for each row in the matrix;
computing a row standard deviation for each row in the matrix; and
for each value, subtracting the row average and dividing by the row standard deviation.

22-47. (canceled)

48. The computer-implemented method of claim 1, wherein each of the single nucleotide variants is a heterozygous variant.

49. The computer-implemented method of claim 1, wherein the computing the reduced distance may comprise one or more of the following: scaling linearly, scaling using a nonlinear function, or binning.

50. (canceled)

51. (canceled)

52. The computer-implemented method of either claim 48, wherein the filtering comprises filtering the SNVs to consider variant quality.

53-72. (canceled)

73. A computer-implemented method of generating a representation of a genome, the method comprising:
identifying, for each single nucleotide variant (SNV) observed in a portion of the genome, a location of the SNV; and
for each consecutive pair of SNV locations:
computing a distance between the respective locations of the pair of SNVs;
computing a reduced distance; and
incrementing a counting value corresponding to the reduced distance.

74. The computer-implemented method of claim 73, further comprising choosing a mask for each pair key, wherein the mask assigns a class value to each counting value corresponding to both the pair key and the reduced distance.

75. The computer-implemented method of claim 74, wherein the class value is one of the following values: 0 or 1.

76-81. (canceled)

82. A computer-implemented method of generating a representation of a portion of a genome, the method comprising:
identifying a plurality of distance values in the portion of the genome;
creating a first reduced representation of the portion of the genome by, for each of the distance values:
computing a first reduced distance, wherein computing the first reduced distance comprises finding the remainder after division of the respective distance value by a first vector length, n1; and
incrementing a counting value according to at least the first reduced distance;
creating a second reduced representation of the portion of the genome by, for each of the distance values:
computing a second reduced distance, wherein computing the second reduced distance comprises finding the remainder after division of the respective distance value by a second vector length, n2; and
incrementing a counting value according to at least the second reduced distance;
normalizing the first and second reduced representations of the portion of the genome to create, respectively, first and second normalized reduced representations;
joining the first and second normalized reduced representations of the portion of the genome to create the representation of the portion of the genome.

83. The method of claim 82, wherein each of the distance values corresponds to the distance between a set of consecutive SNVs observed in the portion of the genome.

84. The method of claim 82, wherein
each of the distance values corresponds to the distance between consecutive locations exhibiting heterozygosity.

85-114. (canceled)
